(19) United States
(12) Patent Application Publication
(10) Pub. No.: US 2012/0003648 A1
(43) Pub. Date: Jan. 5, 2012

(54) Signal Multiplexing and Signal Amplification

(75) Inventors: Yunqing Ma, San Jose, CA (US); Gary K. McMaster, Ann Arbor, MI (US); Aiguo Zhang, San Ramon, CA (US); Quan N. Nguyen, San Ramon, CA (US)

(73) Assignee: Affymetrix, INC., Santa Clara, CA (US)

(21) Appl. No.: 13/175,393

(22) Filed: Jul. 1, 2011

Related U.S. Application Data
(60) Provisional application No. 61/360,912, filed on Jul. 1, 2010.

(51) Int. Cl. C12Q 1/68

(52) U.S. Cl. 435/6.11

(57) ABSTRACT

Disclosed are methods, compositions and kits for amplifying signals for detecting the presence, quantity and/or sequence of nucleic acids and proteins, as well as methods, compositions and kits for increasing the number of such targets simultaneously detectable in a sample. Detection may be, for instance, in vivo, in cellulo or in situ. Amplification of signal is achieved by way of hybridization of nucleic acid label probe systems and structures. Increase in target multiplex capacity is achieved by way of varying the type of labels utilized in the nucleic acid label probe system.

### RELATED APPLICATIONS

The present application claims priority to U.S. Provisional Patent Application Ser. No. 61/360,912, filed on Jul. 1, 2010, the entire disclosure of which is incorporated herein by reference for all purposes.

### FIELD OF THE INVENTION

Disclosed are methods, compositions and kits related to nucleic acid and protein detection in assays.

### BACKGROUND OF THE INVENTION

Many methods, systems and reagents exist which are designed to detect minute quantities of nucleic acid and/or protein targets. However, most such systems require time-consuming and difficult manipulation steps, such as amplification of nucleic acid targets using polymerase enzymes and the like. Other methods utilizing microarrays are also available which allow detection and sequencing of nucleic acids. However, recent advances have made these approaches obsolete. For instance, a product called QUANTIGENE® (Panomics, Fremont, Calif.), is able to specifically bind and detect dozens of molecular targets in a single sample. See, for instance, U.S. patent application Ser. Nos. 11/433,081 (allowed), 11/431,092, 11/471,025 (allowed), all of which are incorporated herein by reference in their entirety for all purposes. General protocols and user's guides on how the QUANTIGENE® system works and explanation of kits and components may be found at the Panomics website (see, (www.)panomics.com/index.php?id=product_{—}1#product_lit_{—}1). Specifically, user's manual, “QUANTIGENE® 2.0 Reagent System User Manual,” (2007) provided at the Panomics website is incorporated herein by reference in its entirety for all purposes (see, panomics.com/downloads/UM13074_QG2Manual_RevB_{—}080102.pdf and other addendums and FFPE Method Proficiency Kit User Manual and addendums also available from the Panomics website, for instance as the User Manual for FFPE available here: panomics.com/downloads/UM13898_QGFFPEProfManual_RevC_{—}071209.pdf, all of which are specifically incorporated herein by reference in their entirety for all purposes).

QUANTIGENE® technology allows unparalleled signal amplification capabilities that provide an extremely sensitive assay. For instance, it is commonly claimed that the limit of detection in situ for mRNA species is about 20 copies of message per cell. However, in practice the limit of detection, due to the variability in the assay, is generally found to be around 50-60 copies of message per cell. This limit of detection limits the field of research since 80% of mRNAs are present at fewer than 5 copies per cell and 95% of mRNAs are present in cells at fewer than 50 copies per cell. As mentioned above, to arrive at this sensitivity, other approaches are very time consuming and complicated. Other technologies rely on the use of a panel of various enzymes and are affected by the fixation process of FFPE. In contrast, the QUANTIGENE® technology, such as QUANTIGENE® 2.0 and ViewRNA, is very simple, efficient and is capable of applying up to 400 labels per 50 base pairs of target. This breakthrough technology allows efficient and simple detection on the level of even a single mRNA copy per cell. Coupling this technology to detection of both mRNA and protein species will propel this field of research into heretofor inaccessible areas of study.

Many different avenues of research have been investigated to address the issues of specificity and sensitivity of hybridization-based genetic assays. For instance, the use of oligonucleotide analogs have been investigated which increase the melting temperature at which the target hybridizes to the capture oligonucleotide.

Improved methods for hybridizing oligonucleotide probes in a specific manner with high affinity and high sensitivity to target nucleic acids and proteins are thus desirable. Among other aspects, presently disclosed are methods, compositions and kits that address these limitations and which permit rapid, simple, and highly specific capture of multiple nucleic acid and protein targets simultaneously.

### SUMMARY OF THE INVENTION

Methods, compositions and kits suitable for amplifying a nucleic acid detection signal are disclosed. Briefly, the methods comprise hybridizing one or more label extender probes to a target nucleic acid, hybridizing a pre-amplifier to the one or more label extender probes, hybridizing one or more amplifiers to the pre-amplifier, hybridizing one or more label spoke probes to the one or more amplifiers, and hybridizing one or more label probes to the one or more label spoke probes. The label probes may be distinguishably different label probes and the sample may comprise multiple target nucleic acids, such that each target nucleic acid has hybridized to it a specific distinguishably different label. The one or more label extender probes comprise one or more nucleic acid analogs, such as the cEt analogs. The assay may optionally comprise hybridizing one or more capture extender probes with the target nucleic acid, and hybridizing the one or more capture extenders to one or more capture probes, wherein the capture probes are covalently attached to a substrate. Compositions and kits comprise all of the various components of the label probe systems mentioned above.

Further, the methods may utilize one or more substrates of varying size, shape and color, each being associated with a different target nucleic acid. The assays described herein may be conducted in situ, in cellulo or in vitro.

The sample to be analyzed may contact target proteins and target nucleic acids, and mixtures thereof. Using the same compositions, kits and methods, all of the desired targets may be detected simultaneously or in series. Detection of target proteins is achieved by hybridizing to the target protein(s) a molecule possessing affinity for the target protein and possessing covalently conjugated thereto a pre-amplifier probe, hybridizing one or more amplifiers to the pre-amplifier, hybridizing one or more label spoke probes to the one or more amplifiers, and hybridizing one or more label probes to the one or more label spoke probes. The label probes may be distinguishably different label probes and the sample may comprise multiple target proteins, such that each target nucleic acid has hybridized to it a specific distinguishably different label. The assay may optionally comprise immobilization of the target proteins to a substrate, before or after hybridization of the label probe system. Compositions and kits comprise all of the various components of the label probe systems mentioned above. Further, these methods may utilize one or more substrates of varying size, shape and color, each being associated with a different target nucleic acid. The assays described herein may be conducted in situ, in cellulo or in vitro.

Essentially all of the features noted for the embodiments above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

### BRIEF DESCRIPTION OF THE DRAWINGS

FIG. 1 schematically illustrates a typical standard bDNA assay.

FIG. 2, Panels A-E schematically depict a multiplex nucleic acid detection assay, in which the nucleic acids of interest are captured on distinguishable subsets of microspheres and then detected.

FIG. 3, Panels A-D schematically depict an embodiment of a multiplex nucleic acid detection assay, in which the nucleic acids of interest are captured at selected positions on a solid support and then detected. Panel A shows a top view of the solid support, while Panels B-D show the support in cross-section.

FIG. 4, Panel A schematically depicts a double Z label extender configuration. Panel B schematically depicts a cruciform label extender configuration.

FIG. 5A schematic of amplification multimer complex and labeling system for a cruciform structure label extender design. Note that in this non-limiting depiction, as in others provided herein, only provides a single example of amplifier/pre-amplifier complex. In the assays, more or fewer amplifiers and label probes may be employed as needed.

FIG. 5B schematic of amplification multimer complex and labeling system for a “double z” or ZZ structure label extender design. Note that in this non-limiting depiction, as in others provided herein, only provides a single example of amplifier/pre-amplifier complex. In the assays, more or fewer amplifiers and label probes may be employed as needed.

FIG. 6A depiction of a locked nucleic acid analog known as the constrained ethyl (cEt) nucleic acid analog. Note that as depicted various protecting groups known in the art are presented but may be substituted by any number of suitable protecting groups.

FIG. 6B depiction of a generic locked nucleic acid analog in the β-D, C3′-endo, conformation. The letter “B” stands for “base” which may be any one of A, G, C, mC, T or U. The methylene bridge connecting the 2′-O atom with the 4′-C atom is the chemical structure which “locks” the analog into the energy-favorable β-D conformation. However, it is understood that this bridge may be any number of carbon atoms in length and may contain any number of variable groups or substitutions as has been reported in the literature Note that as depicted various protecting groups known in the art are presented but may be substituted by any number of suitable protecting groups.

FIG. 7A depiction of single-stranded target SNP detection embodiments utilizing the cruciform (left panel) and the double Z (right panel) structures for the label extenders.

FIG. 7B depiction of double-stranded (dsDNA) target SNP detection embodiments utilizing the cruciform (left panel) and the double Z (right panel) structures for the label extenders.

FIG. 8A depicts various non-limiting conformations and geometries of label extender (LE) probes for detecting single stranded nucleic acid species. Other stereoisomers, conformers and various conformations are possible which achieve similar results but may not be depicted here. Note that for convenience the amplifiers and pre-amplifiers and label probes are not fully represented for all figures. The single line in light shading labeled as “label probe system” is meant to denote all possible configurations of label probe structures as depicted in FIGS. 6A, 6B, 12A and 12B.

FIG. 8B depicts various non-limiting conformations and geometries of label extender (LE) probes for detecting double-stranded nucleic acid species (ability to distinguish between double-stranded DNA targets and ssDNA or RNA targets). Other stereoisomers, conformers and various conformations are possible which achieve similar results but may not be depicted here. Note that for convenience the amplifiers and pre-amplifiers and label probes are not fully represented for all figures. The single line in light shading labeled as “label probe system” is meant to denote all possible suitable configurations of label probe structures.

FIGS. 9A and 9B depict directionality of various label extenders and the possibility that label extenders may be designed in either direction as indicated.

FIG. 10 schematically illustrates signal amplification using pre-amplifier probes, amplifier probes, label spoke probes and label probes.

FIG. 11 schematically illustrates the various multiplexing capabilities achievable in light of the presently disclosed embodiments.

Schematic figures are not necessarily to scale.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a molecule” includes a plurality of such molecules, and the like.

The term “about” as used herein indicates the value of a given quantity varies by +/−10% of the value, or optionally +/−5% of the value, or in some embodiments, by +/−1% of the value so described.

The term “antibody” as referred to herein includes whole antibodies and any antigen binding fragment (i.e., “antigen-binding portion”) or single chains thereof. The term is meant to encompass all known isotypes of antibody, such as, for instance, IgG, IgA, IgD, IgE, and IgM. An “antibody” refers to a glycoprotein comprising at least two heavy (H) chains and two light (L) chains inter-connected by disulfide bonds, or an antigen binding portion thereof. The V_{H} and V_{L} regions of antibodies can be subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. That is, the term antibody is meant to encompass whole antibodies and fragments thereof that possess antigenic binding capability, such as, but not limited to, minibodies, diabodies, triabodies, tetrabodies, and the like. (See, for instance, Olafsen et al., Prot. Eng. Design and Selection, 17(4):315-323, 2004, Tramontano et al., J. Mol. Recognit., 7(1):9-24, 1994, and Todorovska et al., J. Immunol. Methods, 248(1-2):47-66, 2001). Furthermore, the term antibody is meant to encompass humanized antibodies or otherwise engineered antibodies which possess the desired antigen binding activity.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a F_{ab} fragment, a monovalent fragment consisting of the V_{L}, V_{H}, C_{L} and C_{H1} domains; (ii) a F(ab′)₂ fragment, a bivalent fragment comprising two F_{ab} fragments linked by a disulfide bridge at the hinge region; (iii) a F_{d} fragment consisting of the V_{H} and C_{H1} domains; (iv) a Fᵥ fragment consisting of the V_{L} and V_{H} domains of a single arm of an antibody, (v) a dAb fragment (Ward et al., Nature, 341:544-546, 1989), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fᵥ (scFv); see e.g., Bird et al., Science, 242:423-426, 1988; and Huston et al., Proc. Natl. Acad. Sci. USA, 85:5879-5883, 1988). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “polynucleotide” (and the equivalent term “nucleic acid”) encompasses any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

The term “analog” in the context of nucleic acid analog is meant to denote any of a number of known nucleic acid analogs such as, but not limited to, LNA, PNA, etc. For instance, it has been reported that LNA, when incorporated into oligonucleotides, exhibit an increase in the duplex melting temperature of 2° C. to 8° C. per analog incorporated into a single strand of the duplex. The melting temperature effect of incorporated analogs may vary depending on the chemical structure of the analog, e.g. the structure of the atoms present in the bridge between the 2′-O atom and the 4′-C atom of the ribose ring of a nucleic acid.

Examples of issued US Patents and Published U.S. Patent Applications disclosing various bicyclic nucleic acids include, for example, U.S. Pat. Nos. 6,770,748, 6,268,490 and 6,794,499 and U.S. Patent Application Publication Nos. 20040219565, 20040014959, 20030207841, 20040192918, 20030224377, 20040143114, 20030087230 and 20030082807, the text of each of which is incorporated by reference herein, in their entirety.

As additional examples, various 5′-modified nucleosides have also been reported. (See, for example: Mikhailov et al., Nucleosides and Nucleotides, 1991, 10:393-343; Saha et al., J. Org. Chem., 1995, 60:788-789; Beigleman et al., Nucleosides and Nucleotides, 1995, 14:901-905; Wang, et al., Bioorganic & Medicinal Chemistry Letters, 1999, 9:885-890; and PCT Internation Application Number WO94/22890 which was published Oct. 13, 1994, the text of each of which is incorporated by reference herein, in their entirety).

Oligonucleotides in solution as single stranded species rotate and move in space in various energy-minimized conformations. Upon binding and ultimately hybridizing to a complementary sequence, an oligonucleotide is known to undergo a conformational transition from the relatively random coil structure of the single stranded state to the ordered structure of the duplex state. With these physical-chemical dynamics in mind, a number of conformationally-restricted oligonucleotides analogs, including bicyclic and tricyclic nucleoside analogues, have been synthesized, incorporated into oligonucleotides and tested for their ability to hybridize. It has been found that various nucleic acid analogs, such as the common “Locked Nucleic Acid” or LNA, exhibit a very low energy-minimized state upon hybridizing to the complementary oligonucleotide, even when the complementary oligonucleotide is wholly comprised of the native or natural nucleic acids A, T, C, U and G.

Some U.S. patents have disclosed various modifications of these analogs that exhibit the desired properties of being stably integrated into oligonucleotide sequences and increasing the melting temperature at which hybridization occurs, thus producing a very stable, energy-minimized duplex with oligonucleotides comprising even native nucleic acids. (See, for instance, U.S. Pat. Nos. 7,572,582, 7,399,845, 7,034,133, 6,794,499 and 6,670,461, all of which are incorporated herein by reference in their entirety for all purposes). For instance, U.S. Pat. No. 7,399,845 provides 6-modified bicyclic nucleosides, oligomeric compounds and compositions prepared therefrom, including novel synthetic intermediates, and methods of preparing the nucleosides, oligomeric compounds, compositions, and novel synthetic intermediates. The '845 patent discloses nucleosides having a bridge between the 4′ and 2′-positions of the ribose portion having the formula: 2′-O—C(H)(Z)-4′ and oligomers and compositions prepared therefrom. In a preferred embodiment, Z is in a particular configuration providing either the (R) or (S) isomer, e.g. 2′-O,4′-methanoribonucleoside. It was shown that this nucleic acid analog exists as the strictly constrained N-conformer 2′-exo-3′-endo conformation. Oligonucleotides of 12 nucleic acids in length have been shown, when comprised completely or partially of the Imanishi et al. analogs, to have substantially increased melting temperatures, showing that the corresponding duplexes with complementary native oligonucleotides are very stable. (See, Imanishi et al., “Synthesis and property of novel conformationally constrained nucleoside and oligonucleotide analogs,” The Sixteenth International Congress of Heterocyclic Chemistry, Aug. 10-15, 1997, incorporated herein by reference in its entirety for all purposes).

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

Two polynucleotides “hybridize” when they associate to form a stable duplex, e.g., under relevant assay conditions. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York), as well as in Ausubel, infra.

The “Tₘ” (melting temperature) of a nucleic acid duplex under specified conditions (e.g., relevant assay conditions) is the temperature at which half of the base pairs in a population of the duplex are disassociated and half are associated. The Tₘ for a particular duplex can be calculated and/or measured, e.g., by obtaining a thermal denaturation curve for the duplex (where the Tₘ is the temperature corresponding to the midpoint in the observed transition from double-stranded to single-stranded form).

The term “complementary” refers to a polynucleotide that forms a stable duplex with its “complement,” e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

A “capture extender” or “CE” is a polynucleotide that is capable of hybridizing to a nucleic acid of interest and to a capture probe. The capture extender typically has a first polynucleotide sequence C-1, which is complementary to the capture probe, and a second polynucleotide sequence C-3, which is complementary to a polynucleotide sequence of the nucleic acid of interest. Sequences C-1 and C-3 are typically not complementary to each other. The capture extender is preferably single-stranded.

A “capture probe” or “CP” is a polynucleotide that is capable of hybridizing to at least one capture extender and that is tightly bound (e.g., covalently or noncovalently, directly or through a linker, e.g., streptavidin-biotin or the like) to a solid support, a spatially addressable solid support, a slide, a particle, a microsphere, or the like. The capture probe typically comprises at least one polynucleotide sequence C-2 that is complementary to polynucleotide sequence C-1 of at least one capture extender. The capture probe is preferably single-stranded.

A “label extender” or “LE” is a polynucleotide that is capable of hybridizing to a nucleic acid of interest and to a label probe system. The label extender typically has a first polynucleotide sequence L-1, which is complementary to a polynucleotide sequence of the nucleic acid of interest, and a second polynucleotide sequence L-2, which is complementary to a polynucleotide sequence of the label probe system (e.g., L-2 can be complementary to a polynucleotide sequence of an amplification multimer, a preamplifier, a label probe, or the like). The label extender is preferably single-stranded. Label extenders designed in both directions are contemplated, i.e. a label extender in the 3′ to 5′ direction could just as easily be designed to bind in the reverse direction as depicted in the Figures. For instance, see FIGS. 12A and 12B for exemplary depictions of the various configurations which may be designed to be suitable for use in the presently disclosed invention.

A “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Many labels are commercially available and can be used in the context of the invention.

A “label probe system” comprises one or more polynucleotides that collectively comprise a label and at least two polynucleotide sequences M-1, each of which is capable of hybridizing to a label extender. The label provides a signal, directly or indirectly. Polynucleotide sequence M-1 is typically complementary to sequence L-2 in the label extenders. The at least two polynucleotide sequences M-1 are optionally identical sequences or different sequences. The label probe system can include a plurality of label probes (e.g., a plurality of identical label probes) and an amplification multimer; it optionally also includes a preamplifier or the like, or optionally includes only label probes, for example.

An “amplification multimer” is a polynucleotide comprising a plurality of polynucleotide sequences M-2, typically (but not necessarily) identical polynucleotide sequences M-2. Polynucleotide sequence M-2 is complementary to a polynucleotide sequence in the label probe. The amplification multimer also includes at least one polynucleotide sequence that is capable of hybridizing to a label extender or to a nucleic acid that hybridizes to the label extender, e.g., a preamplifier. For example, the amplification multimer optionally includes at least one (and preferably at least two) polynucleotide sequence(s) M-1, optionally identical sequences M-1; polynucleotide sequence M-1 is typically complementary to polynucleotide sequence L-2 of the label extenders. Similarly, the amplification multimer optionally includes at least one polynucleotide sequence that is complementary to a polynucleotide sequence in a preamplifier. The amplification multimer can be, e.g., a linear or a branched nucleic acid. That is, the amplification multimer may be entirely comprised of a single contiguous chain of nucleic acids, or alternative a first chain possessing the sequence M-1 and additionally possessing one more sequences A-1 that are complementary to sequences A-2 on separate oligonucleotides which comprise one or more repeats of the sequence M-2. Thus, the amplification multimer may in fact be an assembly of multiple oligonucleotides comprising or consisting of a pre-amplifier possessing the M-2 sequence and one or more A-1 sequences; and one or more amplifier oligonucleotides possessing the sequence A-2 and one or more sequences M-2. Upon hybridization the structure may yield a tree-like geometrical shape comprising a single pre-amplifier, multiple amplifiers and attached to the amplifiers, multiple label probes which hybridize to site(s) M-2. As noted for all polynucleotides, the amplification multimer can include modified nucleotides and/or nonstandard internucleotide linkages as well as standard deoxyribonucleotides, ribonucleotides, and/or phosphodiester bonds. Suitable amplification multimers are described, for example, in U.S. Pat. No. 5,635,352, U.S. Pat. No. 5,124,246, U.S. Pat. No. 5,710,264, and U.S. Pat. No. 5,849,481.

A “label probe” or “LP” is a single-stranded polynucleotide that comprises a label (or optionally that is configured to bind to a label) that directly or indirectly provides a detectable signal. The label probe typically comprises a polynucleotide sequence that is complementary to the repeating polynucleotide sequence M-2 of the amplification multimer; however, if no amplification multimer is used in the bDNA assay, the label probe can, e.g., hybridize directly to a label extender.

A “preamplifier” is a nucleic acid that serves as an intermediate between one or more label extenders and amplifiers. Typically, the preamplifier is capable of hybridizing simultaneously to at least two label extenders and to a plurality of amplifiers.

A “microsphere” is a small spherical, or roughly spherical, particle. A microsphere typically has a diameter less than about 1000 micrometers (e.g., less than about 100 micrometers, optionally less than about 10 micrometers).

“Microparticles” include particles having a code, including sets of encoded microparticles. (See, for instance, U.S. patent application Ser. No. 11/521,057, allowed, which is incorporated herein by reference in its entirety for all purposes). Such encoded microparticles may have a longest dimension of 50 microns, an outer surface substantially of glass and a spatial code that can be read with optical magnification. A microparticle may be cuboid in shape and elongated along the Y direction in the Cartesian coordinate. The cross-sections perpendicular to the length of the microparticle may have substantially the same topological shape—such as square shape. Microparticles may have a set of segments and gaps intervening the segments in parallel along the axis of the longest dimension if the microparticle is rectangular. Specifically, segments with different lengths (the dimension along the length of the microparticle, e.g. along the Y direction) may represent different coding elements; whereas gaps preferably have the same length for differentiating the segments during detection of the microparticles. The segments of the microparticle may be fully enclosed within the microparticle, i.e. completely encapsulated by a surrounding outer layer which may be silicon/glass. As an alternative feature, the segments can be arranged such that the geometric centers of the segments are aligned to the geometric central axis of the elongated microparticle. A particular sequence of segments and gaps thereby represent a code within each micoparticle. The codes may be derived from a pre-determined coding scheme thereby allowing identification of the microparticle. The microparticles may additionally have various structural aberrations, such as tags or tabs, on one or more ends, thus allowing for a two-fold or more increase in code space. The microparticles may also be present as a “bi-particle” wherein the microparticle actually comprises two or more particles stuck together, i.e. missing the last etching step so as to allow two particles to remain attached together with an intervening material between them comprised of material consistent with the coating present on the rest of the microparticle. (See, for instance, U.S. patent application Ser. No. 12/779,413, filed May 13, 2010, incorporated herein by reference in its entirety for all purposes).

A “microorganism” is an organism of microscopic or submicroscopic size. Examples include, but are not limited to, bacteria, fungi, yeast, protozoans, microscopic algae (e.g., unicellular algae), viruses (which are typically included in this category although they are incapable of growth and reproduction outside of host cells), subviral agents, viroids, and mycoplasma.

A first polynucleotide sequence that is located “5′ of” a second polynucleotide sequence on a nucleic acid strand is positioned closer to the 5′ terminus of the strand than is the second polynucleotide sequence. Similarly, a first polynucleotide sequence that is located “3′ of” a second polynucleotide sequence on a nucleic acid strand is positioned closer to the 3′ terminus of the strand than is the second polynucleotide sequence.

A variety of additional terms are defined or otherwise characterized herein.

### DETAILED DESCRIPTION

Presently disclosed are methods, compositions and kits for amplifying signals for detecting the presence, quantity and/or sequence of nucleic acids and proteins, as well as methods, compositions and kits for increasing the number of such targets simultaneously detectable in a sample. Detection may be, for instance, in vivo, in cellulo or in situ. Amplification of signal is achieved by way of hybridization of nucleic acid label probe systems and structures. Increase in target multiplex capacity is achieved by way of varying the type of labels utilized in the nucleic acid label probe system.

A general class of embodiments includes methods of capturing two or more nucleic acids of interest and identification thereof. In the methods, a sample, a pooled population of particles (or microparticles, or encoded microparticles), and two or more subsets of n target capture probes, wherein n is at least two, are provided. The sample comprises or is suspected of comprising the nucleic acids of interest. The pooled population of particles includes two or more subsets of particles. The particles in each subset have associated therewith a different capture probes. Each subset of n capture extenders is capable of hybridizing to one of the nucleic acids of interest, and the capture extenders in each subset are capable of hybridizing to one of the capture probes and thereby associating each subset of n target capture probes with a selected subset of the particles. Preferably, a plurality of the particles in each subset is distinguishable from a plurality of the particles in every other subset. (Typically, substantially all of the particles in each subset are distinguishable from substantially all of the particles in every other subset.) Each nucleic acid of interest can thus, by hybridizing to its corresponding subset of n capture extenders which are in turn hybridized to a corresponding capture probes, be associated with an identifiable subset of the particles. Alternatively, the particles in the various subsets need not be distinguishable from each other (for example, in embodiments in which any nucleic acid of interest present is to be isolated, amplified, and/or detected, without regard to its identity, following its capture on the particles.)

In one embodiment of the following methodologies and compositions, a particular nucleic acid of interest, or target oligonucleotide, may be captured to a surface through cooperative hybridization of multiple target capture probes to the nucleic acid. Each of the capture extenders (CE) has a first polynucleotide sequence that can hybridize to the target nucleic acid and a second polynucleotide sequence that can hybridize to a complementary sequence on a capture probe that is bound to a surface. The temperature and the stability of the complex between a single CE and its CP can be controlled such that binding of a single CE to a target nucleic acid and to the CP is not sufficient to stably capture the nucleic acid on the surface to which the CP is bound, whereas simultaneous binding of two or more CEs to a target nucleic acid can capture it on the surface vie the two or more CPs. Assays requiring such cooperative hybridization of multiple target capture probes for capture of each nucleic acid of interest results in high specificity and low background from cross-hybridization of the target capture probes with other, non-target nucleic acids. Such low background and minimal cross-hybridization are typically substantially more difficult to achieve in multiplex than a single-plex capture of nucleic acids, because the number of potential nonspecific interactions are greatly increased in a multiplex experiment due to the increased number of probes used (e.g., the greater number of target capture probes). Requiring multiple simultaneous CE-CP interactions for the capture of a target nucleic acid minimizes the chance that nonspecific capture will occur, even when some nonspecific target-CE and/or CE-CP interactions occur.

Branched-chain DNA (bDNA) signal amplification technology has been used, e.g., to detect and quantify mRNA transcripts in cell lines and to determine viral loads in blood. (See, for instance, Player et al. (2001) “Single-copy gene detection using branched DNA (bDNA) in situ hybridization,” J. Histochem. Cytochem., 49:603-611, Van Cleve et al., Mol. Cell. Probes, (1998) 12:243-247, and U.S. Pat. No. 7,033,758, each of which is incorporated herein by reference in their entirety for all purposes). The bDNA assay is a sandwich nucleic acid hybridization procedure that enables direct measurement of mRNA expression, e.g., from crude cell lysate. It provides direct quantification of nucleic acid molecules at physiological levels. Several advantages of the technology distinguish it from other DNA/RNA amplification technologies, including linear amplification, good sensitivity and dynamic range, great precision/specificity and accuracy, simple sample preparation procedure, and reduced sample-to-sample variation.

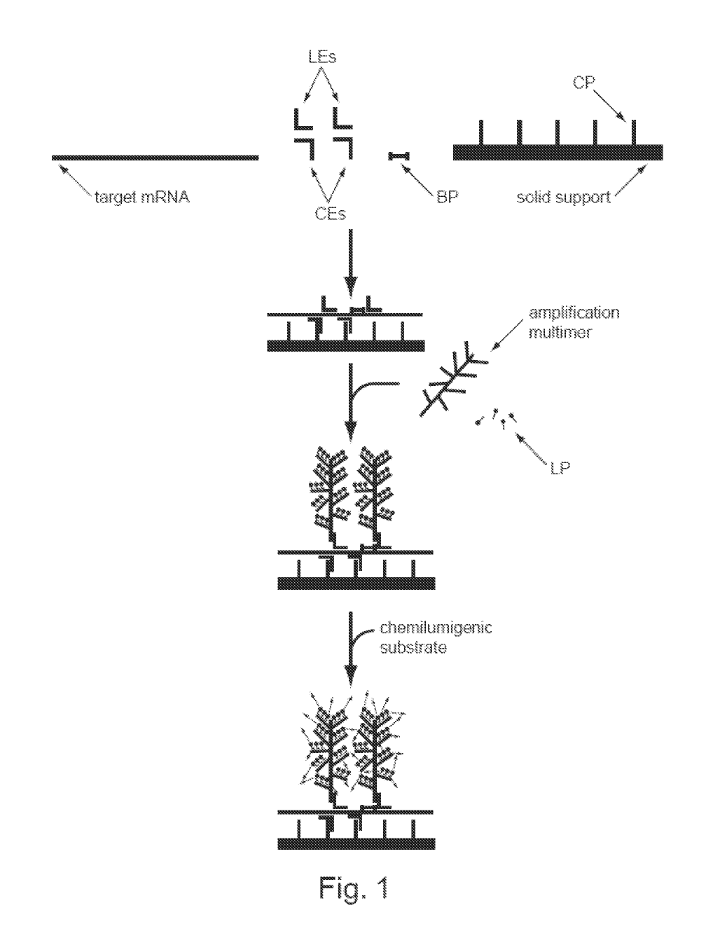

In brief, in a typical bDNA assay for gene expression analysis (FIG. 1), a target mRNA whose expression is to be detected is released from cells and captured by a Capture Probe (CP) on a solid surface (e.g., a well of a microtiter plate) through synthetic oligonucleotide probes called Capture Extenders (CEs). Each capture extender has a first polynucleotide sequence that can hybridize to the target mRNA and a second polynucleotide sequence that can hybridize to the capture probe. Typically, two or more capture extenders are used. Probes of another type, called Label Extenders (LEs), hybridize to different sequences on the target mRNA and to sequences on an amplification multimer. Additionally, Blocking Probes (BPs), which hybridize to regions of the target mRNA not occupied by CEs or LEs, are often used to reduce non-specific target probe binding. A probe set for a given mRNA thus consists of CEs, LEs, and optionally BPs for the target mRNA. The CEs, LEs, and BPs are complementary to nonoverlapping sequences in the target mRNA, and are typically, but not necessarily, contiguous.

Signal amplification begins with the binding of the LEs to the target mRNA. An amplification multimer is then typically hybridized to the LEs. The amplification multimer has multiple copies of a sequence that is complementary to a label probe (it is worth noting that the amplification multimer is typically, but not necessarily, a branched-chain nucleic acid; for example, the amplification multimer can be a branched, forked, or comb-like nucleic acid or a linear nucleic acid). A label, for example, alkaline phosphatase, is covalently attached to each label probe. (Alternatively, the label can be noncovalently bound to the label probes.) In the final step, labeled complexes are detected, e.g., by the alkaline phosphatase-mediated degradation of a chemilumigenic substrate, e.g., dioxetane. Luminescence is reported as relative light unit (RLUs) on a microplate reader. The amount of chemiluminescence is proportional to the level of mRNA expressed from the target gene.

In the preceding example, the amplification multimer and the label probes comprise a label probe system. In another example, the label probe system also comprises a preamplifier, e.g., as described in U.S. Pat. No. 5,635,352 and U.S. Pat. No. 5,681,697, which further amplifies the signal from a single target mRNA. In yet another example, the label extenders hybridize directly to the label probes and no amplification multimer or preamplifier is used, so the signal from a single target mRNA molecule is only amplified by the number of distinct label extenders that hybridize to that mRNA.

Basic bDNA assays have been well described. See, e.g., U.S. Pat. No. 4,868,105 to Urdea et al. entitled “Solution phase nucleic acid sandwich assay”; U.S. Pat. No. 5,635,352 to Urdea et al. entitled “Solution phase nucleic acid sandwich assays having reduced background noise”; U.S. Pat. No. 5,681,697 to Urdea et al. entitled “Solution phase nucleic acid sandwich assays having reduced background noise and kits therefor”; U.S. Pat. No. 5,124,246 to Urdea et al. entitled “Nucleic acid multimers and amplified nucleic acid hybridization assays using same”; U.S. Pat. No. 5,624,802 to Urdea et al. entitled “Nucleic acid multimers and amplified nucleic acid hybridization assays using same”; U.S. Pat. No. 5,849,481 to Urdea et al. entitled “Nucleic acid hybridization assays employing large comb-type branched polynucleotides”; U.S. Pat. No. 5,710,264 to Urdea et al. entitled “Large comb type branched polynucleotides”; U.S. Pat. No. 5,594,118 to Urdea and Horn entitled “Modified N-4 nucleotides for use in amplified nucleic acid hybridization assays”; U.S. Pat. No. 5,093,232 to Urdea and Horn entitled “Nucleic acid probes”; U.S. Pat. No. 4,910,300 to Urdea and Horn entitled “Method for making nucleic acid probes”; U.S. Pat. No. 5,359,100; U.S. Pat. No. 5,571,670; U.S. Pat. No. 5,614,362; U.S. Pat. No. 6,235,465; U.S. Pat. No. 5,712,383; U.S. Pat. No. 5,747,244; U.S. Pat. No. 6,232,462; U.S. Pat. No. 5,681,702; U.S. Pat. No. 5,780,610; U.S. Pat. No. 5,780,227 to Sheridan et al. entitled “Oligonucleotide probe conjugated to a purified hydrophilic alkaline phosphatase and uses thereof”; U.S. patent application Publication No. US2002172950 by Kenny et al. entitled “Highly sensitive gene detection and localization using in situ branched-DNA hybridization”; Wang et al. (1997) “Regulation of insulin preRNA splicing by glucose” Proc Nat Acad Sci USA 94:4360-4365; Collins et al. (1998) “Branched DNA (bDNA) technology for direct quantification of nucleic acids: Design and performance” in Gene Quantification, F Ferre, ed.; and Wilber and Urdea (1998) “Quantification of HCV RNA in clinical specimens by branched DNA (bDNA) technology” Methods in Molecular Medicine: Hepatitis C 19:71-78. In addition, kits for performing basic bDNA assays (QUANTIGENE® kits, comprising instructions and reagents such as amplification multimers, alkaline phosphatase labeled label probes, chemilumigenic substrate, capture probes immobilized on a solid support, and the like) are commercially available, e.g., from Panomics, Inc. (on the world wide web at (www.panomics.com). General protocols and user's guides on how the QUANTIGENE® system works and explanation of kits and components may be found at the Panomics website (see, www.panomics.com/index.php?id=product_{—}1#product_lit_{—}1). Specifically, user's manual, “QUANTIGENE® 2.0 Reagent System User Manual,” (2007, 32 pages) provided at the Panomics website is incorporated herein by reference in its entirety for all purposes. Software for designing probe sets for a given mRNA target (i.e., for designing the regions of the CEs, LEs, and optionally BPs that are complementary to the target) is also commercially available (e.g., ProbeDesigner™ from Panomics, Inc.; see also Bushnell et al. (1999) “ProbeDesigner: for the design of probe sets for branched DNA (bDNA) signal amplification assays Bioinformatics 15:348-55).

The basic bDNA assay, however, permits detection of only a single target nucleic acid per assay, while, as described above, detection of multiple nucleic acids is frequently desirable.

Among other aspects, the present invention provides multiplex bDNA assays that can be used for simultaneous detection of two or more target nucleic acids. Similarly, one aspect of the present invention provides bDNA assays, singleplex or multiplex, that have reduced background from nonspecific hybridization events.

Among other aspects, the present invention provides a multiplex bDNA assay that can be used for simultaneous detection of two or more target nucleic acids. The assay temperature and the stability of the complex between a single CE and its corresponding CP can be controlled such that binding of a single CE to a nucleic acid and to the CP is not sufficient to stably capture the nucleic acid on the surface to which the CP is bound, whereas simultaneous binding of two or more CEs to a nucleic acid can capture it on the surface. Requiring such cooperative hybridization of multiple CEs for capture of each nucleic acid of interest results in high specificity and low background from cross-hybridization of the CEs with other, non-target nucleic acids. For an assay to achieve high specificity and sensitivity, it preferably has a low background, resulting, e.g., from minimal cross-hybridization. Such low background and minimal cross-hybridization are typically substantially more difficult to achieve in a multiplex assay than a single-plex assay, because the number of potential nonspecific interactions are greatly increased in a multiplex assay due to the increased number of probes used in the assay (e.g., the greater number of CEs and LEs). Requiring multiple simultaneous CE-CP interactions for the capture of a target nucleic acid minimizes the chance that nonspecific capture will occur, even when some nonspecific CE-CP interactions do occur.

In general, in the assays of the invention, two or more label extenders are used to capture a single component of the label probe system (e.g., a preamplifier or amplification multimer). The assay temperature and the stability of the complex between a single LE and the component of the label probe system (e.g., the preamplifier or amplification multimer) can be controlled such that binding of a single LE to the component is not sufficient to stably associate the component with a nucleic acid to which the LE is bound, whereas simultaneous binding of two or more LEs to the component can capture it to the nucleic acid. Requiring such cooperative hybridization of multiple LEs for association of the label probe system with the nucleic acid(s) of interest results in high specificity and low background from cross-hybridization of the LEs with other, non-target nucleic acids.

For an assay to achieve high specificity and sensitivity, it preferably has a low background, resulting, e.g., from minimal cross-hybridization. Such low background and minimal cross-hybridization are typically substantially more difficult to achieve in a multiplex assay than a single-plex assay, because the number of potential nonspecific interactions are greatly increased in a multiplex assay due to the increased number of probes used in the assay (e.g., the greater number of CEs and LEs). Requiring multiple simultaneous LE-label probe system component interactions for the capture of the label probe system to a target nucleic acid minimizes the chance that nonspecific capture will occur, even when some nonspecific CE-LE or LE-CP interactions, for example, do occur. This reduction in background through minimization of undesirable cross-hybridization events thus facilitates multiplex detection of the nucleic acids of interest.

The methods of the invention can be used, for example, for multiplex detection of two or more nucleic acids simultaneously, from even complex samples, without requiring prior purification of the nucleic acids, when the nucleic acids are present at low concentration, and/or in the presence of other, highly similar nucleic acids. In one aspect, the methods involve capture of the nucleic acids to particles (e.g., distinguishable subsets of microspheres), while in another aspect, the nucleic acids are captured to a spatially addressable solid support. Compositions, kits, and systems related to the methods are also provided.

### Methods, In General

As noted, one aspect of the invention provides multiplex nucleic acid assays. Thus, one general class of embodiments includes methods of detecting two or more nucleic acids of interest. In one embodiment of the method, a sample comprising or suspected of comprising the nucleic acids of interest, two or more subsets of m label extenders, wherein m is at least two, and a label probe system are provided. Each subset of m label extenders is capable of hybridizing to one of the nucleic acids of interest. The label probe system comprises a label, and a component of the label probe system is capable of hybridizing simultaneously to at least two of the m label extenders in a subset.

Those nucleic acids of interest present in the sample are captured on a solid support. Each nucleic acid of interest captured on the solid support is hybridized to its corresponding subset of m label extenders, and the label probe system is hybridized to the m label extenders. The presence or absence of the label on the solid support is then detected. Since the label is associated with the nucleic acid(s) of interest via hybridization of the label extenders and label probe system, the presence or absence of the label on the solid support is correlated with the presence or absence of the nucleic acid(s) of interest on the solid support and thus in the original sample.

In another embodiment, a sample, a pooled population of particles, and two or more subsets of n capture extenders, wherein n is at least two, are provided. The sample comprises or is suspected of comprising the nucleic acids of interest. The pooled population of particles includes two or more subsets of particles, and a plurality of the particles in each subset are distinguishable from a plurality of the particles in every other subset. (Typically, substantially all of the particles in each subset are distinguishable from substantially all of the particles in every other subset.) The particles in each subset have associated therewith a different capture probe. Each subset of n capture extenders is capable of hybridizing to one of the nucleic acids of interest, and the capture extenders in each subset are capable of hybridizing to one of the capture probes and thereby associating each subset of n capture extenders with a selected subset of the particles. Each nucleic acid of interest can thus, by hybridizing to its corresponding subset of n capture extenders which are in turn hybridized to a corresponding capture probe, be associated with an identifiable subset of the particles.

Essentially any suitable solid support can be employed in the methods. For example, the solid support can comprise particles such as microspheres or microparticles, or it can comprise a substantially planar and/or spatially addressable support. Different nucleic acids are optionally captured on different distinguishable subsets of particles or at different positions on a spatially addressable solid support. The nucleic acids of interest can be captured to the solid support by any of a variety of techniques, for example, by binding directly to the solid support or by binding to a moiety bound to the support, or through hybridization to another nucleic acid bound to the solid support. Preferably, the nucleic acids are captured to the solid support through hybridization with capture extenders and capture probes.

In one class of embodiments, a pooled population of particles which constitute the solid support is provided. The population comprises two or more subsets of particles, and a plurality of the particles in each subset is distinguishable from a plurality of the particles in every other subset. (Typically, substantially all of the particles in each subset are distinguishable from substantially all of the particles in every other subset.) The particles in each subset have associated therewith a different capture probe.

Two or more subsets of n capture extenders, wherein n is at least two, are also provided. Each subset of n capture extenders is capable of hybridizing to one of the nucleic acids of interest, and the capture extenders in each subset are capable of hybridizing to one of the capture probes, thereby associating each subset of n capture extenders with a selected subset of the particles. Each of the nucleic acids of interest present in the sample is hybridized to its corresponding subset of n capture extenders and the subset of n capture extenders is hybridized to its corresponding capture probe, thereby capturing the nucleic acid on the subset of particles with which the capture extenders are associated.

Typically, in this class of embodiments, at least a portion of the particles from each subset are identified and the presence or absence of the label on those particles is detected. Since a correlation exists between a particular subset of particles and a particular nucleic acid of interest, which subsets of particles have the label present indicates which of the nucleic acids of interest were present in the sample.

Essentially any suitable particles, e.g., particles having distinguishable characteristics and to which capture probes can be attached, can be used. For example, in one preferred class of embodiments, the particles are microspheres. The microspheres of each subset can be distinguishable from those of the other subsets, e.g., on the basis of their fluorescent emission spectrum, their diameter, or a combination thereof. For example, the microspheres of each subset can be labeled with a unique fluorescent dye or mixture of such dyes, quantum dots with distinguishable emission spectra, and/or the like. As another example, the particles of each subset can be identified by an optical barcode, unique to that subset, present on the particles.

The particles optionally have additional desirable characteristics. For example, the particles can be magnetic or paramagnetic, which provides a convenient means for separating the particles from solution, e.g., to simplify separation of the particles from any materials not bound to the particles.

In other embodiments, the nucleic acids are captured at different positions on a non-particulate, spatially addressable solid support. Thus, in one class of embodiments, the solid support comprises two or more capture probes, wherein each capture probe is provided at a selected position on the solid support. Two or more subsets of n capture extenders, wherein n is at least two, are provided. Each subset of n capture extenders is capable of hybridizing to one of the nucleic acids of interest, and the capture extenders in each subset are capable of hybridizing to one of the capture probes, thereby associating each subset of n capture extenders with a selected position on the solid support. Each of the nucleic acids of interest present in the sample is hybridized to its corresponding subset of n capture extenders and the subset of n capture extenders is hybridized to its corresponding capture probe, thereby capturing the nucleic acid on the solid support at the selected position with which the capture extenders are associated.

Typically, in this class of embodiments, the presence or absence of the label at the selected positions on the solid support is detected. Since a correlation exists between a particular position on the support and a particular nucleic acid of interest, which positions have a label present indicates which of the nucleic acids of interest were present in the sample.

The solid support typically has a planar surface and is typically rigid, but essentially any spatially addressable solid support can be adapted to the practice of the present invention. Exemplary materials for the solid support include, but are not limited to, glass, silicon, silica, quartz, plastic, polystyrene, nylon, and nitrocellulose. As just one example, an array of capture probes can be formed at selected positions on a glass slide as the solid support.

In any of the embodiments described herein in which capture extenders are utilized to capture the nucleic acids to the solid support, n, the number of capture extenders in a subset, is at least one, preferably at least two, and more preferably at least three. n can be at least four or at least five or more. Typically, but not necessarily, n is at most ten. For example, n can be between three and ten, e.g., between five and ten or between five and seven, inclusive. Use of fewer capture extenders can be advantageous, for example, in embodiments in which nucleic acids of interest are to be specifically detected from samples including other nucleic acids with sequences very similar to that of the nucleic acids of interest. In other embodiments (e.g., embodiments in which capture of as much of the nucleic acid as possible is desired), however, n can be more than 10, e.g., between 20 and 50. n can be the same for all of the subsets of capture extenders, but it need not be; for example, one subset can include three capture extenders while another subset includes five capture extenders. The n capture extenders in a subset preferably hybridize to nonoverlapping polynucleotide sequences in the corresponding nucleic acid of interest. The nonoverlapping polynucleotide sequences can, but need not be, consecutive within the nucleic acid of interest.

Each capture extender is capable of hybridizing to its corresponding capture probe. The capture extender typically includes a polynucleotide sequence C-1 that is complementary to a polynucleotide sequence C-2 in its corresponding capture probe. Capture of the nucleic acids of interest via hybridization to the capture extenders and capture probes optionally involves cooperative hybridization. In one aspect, the capture extenders and capture probes are configured as described in U.S. patent application 60/680,976 filed May 12, 2005 by Luo et al., entitled “Multiplex branched-chain DNA assays.” In one aspect, C-1 and C-2 are 20 nucleotides or less in length. In one class of embodiments, C-1 and C-2 are between 9 and 17 nucleotides in length (inclusive), preferably between 12 and 15 nucleotides (inclusive). For example, C-1 and C-2 can be 14, 15, 16, or 17 nucleotides in length, or they can be between 9 and 13 nucleotides in length (e.g., for lower hybridization temperatures, e.g., hybridization at room temperature).

The capture probe can include polynucleotide sequence in addition to C-2, or C-2 can comprise the entire polynucleotide sequence of the capture probe. For example, each capture probe optionally includes a linker sequence between the site of attachment of the capture probe to the particles and sequence C-2 (e.g., a linker sequence containing 8 Ts, as just one possible example).

It will be evident that the amount of overlap between each individual capture extender and its corresponding capture probe (i.e., the length of C-1 and C-2) affects the Tₘ of the complex between that capture extender and capture probe, as does, e.g., the GC base content of sequences C-1 and C-2. Typically, all the capture probes are the same length (as are sequences C-1 and C-2) from subset of particles to subset, but not necessarily so. Depending, e.g., on the precise nucleotide sequence of C-2, different support capture probes optionally have different lengths and/or different length sequences C-2, to achieve the desired Tₘ. Different support capture probe-target capture probe complexes optionally have the same or different Tₘs.

It will also be evident that the number of capture extenders required for stable capture of a nucleic acid depends, in part, on the amount of overlap between the capture extenders and the capture probe (i.e., the length of C-1 and C-2). For example, if n is 5-7 for a 14 nucleotide overlap, n could be 3-5 for a 15 nucleotide overlap or 2-3 for a 16 nucleotide overlap.

As noted, the hybridizing the subset of n capture extenders to the corresponding support capture probe is performed at a hybridization temperature which is greater than a melting temperature Tₘ of a complex between each individual capture extender and its corresponding capture probe. The hybridization temperature is typically about 5° C. or more greater than the Tₘ, e.g., about 7° C. or more, about 10° C. or more, about 12° C. or more, about 15° C. or more, about 17° C. or more, or even about 20° C. or more greater than the Tₘ.

Stable capture of nucleic acids of interest, e.g., while minimizing capture of extraneous nucleic acids (e.g., those to which n-1 or fewer of the target capture probes bind) can be achieved, for example, by balancing n (the number of target capture probes), the amount of overlap between the capture extenders and the capture probes (the length of C-1 and C-2), and/or the stringency of the conditions under which the target capture probes, the nucleic acids, and the support capture probes are hybridized.

Appropriate combinations of n, amount of complementarity between the capture extenders and the capture probes, and stringency of hybridization can, for example, be determined experimentally by one of skill in the art. For example, a particular value of n and a particular set of hybridization conditions can be selected, while the number of nucleotides of complementarity between the capture extenders and the capture probes is varied until hybridization of the n capture extenders to a nucleic acid captures the nucleic acid while hybridization of a single capture extender does not efficiently capture the nucleic acid. Similarly, n, amount of complementarity, and stringency of hybridization can be selected such that the desired nucleic acid of interest is captured while other nucleic acids present in the sample are not efficiently captured. Stringency can be controlled, for example, by controlling the formamide concentration, chaotropic salt concentration, salt concentration, pH, organic solvent content, and/or hybridization temperature.

For a given nucleic acid of interest, the corresponding target capture probes are preferably complementary to physically distinct, nonoverlapping sequences in the nucleic acid of interest, which are preferably, but not necessarily, contiguous. The Tₘs of the individual capture extender-nucleic acid complexes are preferably greater than the hybridization temperature, e.g., by 5° C. or 10° C. or preferably by 15° C. or more, such that these complexes are stable at the hybridization temperature. Sequence C-3, which is the sequence of the CE which is complementary to the target nucleic acid, for each capture extender is typically (but not necessarily) about 17-35 nucleotides in length, with about 30-70% GC content. Potential capture extender sequences (e.g., potential sequences C-3) are optionally examined for possible interactions with non-corresponding nucleic acids of interest, repetitive sequences (such as polyC or polyT, for example), any detection probes used to detect the nucleic acids of interest, and/or any relevant genomic sequences, for example; sequences expected to cross-hybridize with undesired nucleic acids are typically not selected for use in the target support capture probes. Examination can be, e.g., visual (e.g., visual examination for complementarity), computational (e.g., computation and comparison of percent sequence identity and/or binding free energies; for example, sequence comparisons can be performed using BLAST software publicly available through the National Center for Biotechnology Information on the world wide web at ncbi.nlm.nih.gov), and/or experimental (e.g., cross-hybridization experiments). Capture probe sequences are preferably similarly examined, to ensure that the polynucleotide sequence C-1 complementary to a particular capture probe's sequence C-2 is not expected to cross-hybridize with any of the other capture probes that are to be associated with other subsets of particles.

The methods are useful for multiplex detection of nucleic acids, optionally highly multiplex detection. Thus, the two or more nucleic acids of interest (i.e., the nucleic acids to be detected) optionally comprise five or more, 10 or more, 20 or more, 30 or more, 40 or more, 50 or more, or even 100 or more nucleic acids of interest, while the two or more subsets of m label extenders comprise five or more, 10 or more, 20 or more, 30 or more, 40 or more, 50 or more, or even 100 or more subsets of m label extenders. In embodiments in which capture extenders, particulate solid supports, and/or spatially addressable solid support are used, a like number of subsets of capture extenders, subsets of particles, and/or selected positions on the solid support are provided.

The label probe system optionally includes an amplification multimer and a plurality of label probes, wherein the amplification multimer is capable of hybridizing to the label extenders and to a plurality of label probes. In another aspect, the label probe system includes a preamplifier, a plurality of amplification multimers, and a plurality of label probes, wherein the preamplifier hybridizes to the label extenders, and the amplification multimers hybridize to the preamplifier and to the plurality of label probes. As another example, the label probe system can include only label probes, which hybridize directly to the label extenders. In one class of embodiments, the label probe comprises the label, e.g., a covalently attached label. In other embodiments, the label probe is configured to bind a label; for example, a biotinylated label probe can bind to a streptavidin-associated label.

The label can be essentially any convenient label that directly or indirectly provides a detectable signal. In one aspect, the label is a fluorescent label (e.g., a fluorophore or quantum dot). Detecting the presence of the label on the particles thus comprises detecting a fluorescent signal from the label. In embodiments in which the solid support comprises particles, fluorescent emission by the label is typically distinguishable from any fluorescent emission by the particles, e.g., microspheres, and many suitable fluorescent label-fluorescent microsphere combinations are possible. As other examples, the label can be a luminescent label, a light-scattering label (e.g., colloidal gold particles), or an enzyme (e.g., HRP). Various labels are known in the art, such as Alexa Fluor Dyes (Life Technologies, Inc., California, USA, available in a wide variety of wavelengths, see for instance, Panchuk, et al., J. Hist. Cyto., 47:1179-1188, 1999), biotin-based dyes, digoxigenin, AttoPhos (JBL Scientific, Inc., California, USA, available in a variety of wavelengths, see for instance, Cano et al., Biotechniques, 12(2):264-269, 1992), ATTO dyes (Sigma-Aldrich, St. Louis, Mo.), or any other suitable label.

As noted above, a component of the label probe system is capable of hybridizing simultaneously to at least two of the m label extenders in a subset. Typically, the component of the label probe system that hybridizes to the two or more label extenders is an amplification multimer or preamplifier. Preferably, binding of a single label extender to the component of the label probe system (e.g., the amplification multimer or preamplifier) is insufficient to capture the label probe system to the nucleic acid of interest to which the label extender binds. Thus, in one aspect, the label probe system comprises an amplification multimer or preamplifier, which amplification multimer or preamplifier is capable of hybridizing to the at least two label extenders, and the label probe system (or the component thereof) is hybridized to the m label extenders at a hybridization temperature, which hybridization temperature is greater than a melting temperature Tₘ of a complex between each individual label extender and the amplification multimer or preamplifier. The hybridization temperature is typically about 5° C. or more greater than the Tₘ, e.g., about 7° C. or more, about 10° C. or more, about 12° C. or more, about 15° C. or more, about 17° C. or more, or even about 20° C. or more greater than the Tₘ. It is worth noting that the hybridization temperature can be the same or different than the temperature at which the label extenders and optional capture extenders are hybridized to the nucleic acids of interest.

Each label extender typically includes a polynucleotide sequence L-1 that is complementary to a polynucleotide sequence in the corresponding nucleic acid of interest and a polynucleotide sequence L-2 that is complementary to a polynucleotide sequence in the component of the label probe system (e.g., the preamplifier or amplification multimer). It will be evident that the amount of overlap between each individual label extender and the component of the label probe system (i.e., the length of L-2 and M-1) affects the Tₘ of the complex between the label extender and the component, as does, e.g., the GC base content of sequences L-2 and M-1. Optionally, all the label extenders have the same length sequence L-2 and/or identical polynucleotide sequences L-2. Alternatively, different label extenders can have different length and/or sequence polynucleotide sequences L-2. It will also be evident that the number of label extenders required for stable capture of the component to the nucleic acid of interest depends, in part, on the amount of overlap between the label extenders and the component (i.e., the length of L-2 and M-1).

Stable capture of the component of the label probe system by the at least two label extenders, e.g., while minimizing capture of extraneous nucleic acids, can be achieved, for example, by balancing the number of label extenders that bind to the component, the amount of overlap between the label extenders and the component (the length of L-2 and M-1), and/or the stringency of the conditions under which the label extenders and the component are hybridized. For instance, when detecting a large message RNA of several hundred base pairs or less, any number of label extenders may be used, such as, for instance, 1-30 pairs of label extender probes, or 2-28 pairs of label extender probes, or 3-25 pairs of label extender probes, or 4-20 pairs of label extender probes, or a number of label extender probe pairs which is suitable to specifically attach the label probe system to the target with the desired affinity.

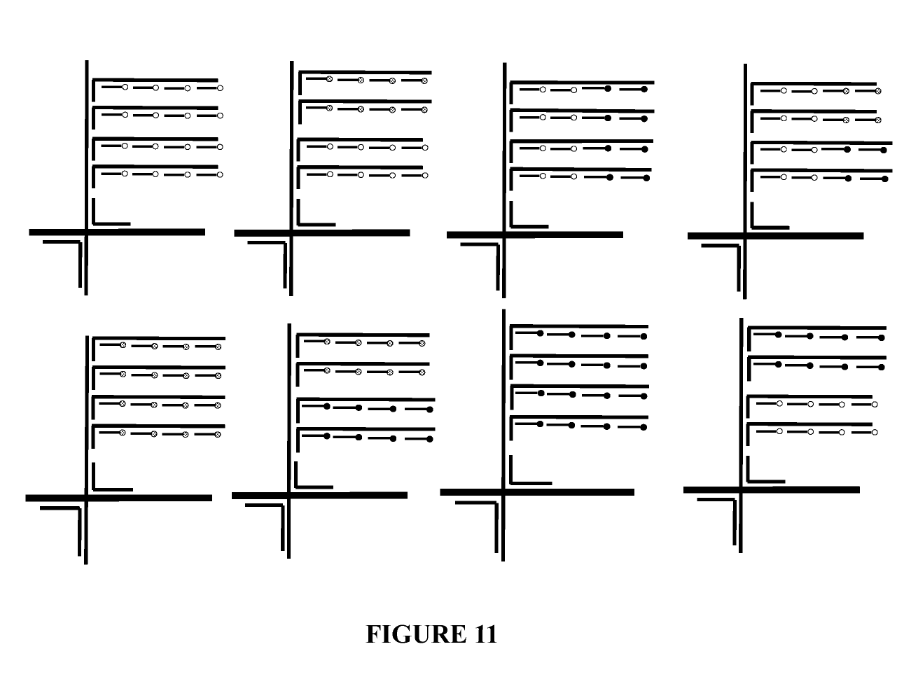

As noted above, while some embodiments generally utilize two label extender probes to hybridize to each pre-amplifier, it is possible in other embodiments to design systems in which three label extender probes hybridize to a single target and single pre-amplifier probe, or even four label extender probes per pre-amplifier. Further, when the target nucleic acid is particularly short, as in siRNA or miRNA, it is possible to use only a single label extender probe, in concert with a single capture extender probe, to detect the target. (See, for instance, FIG. 11). Alternatively, if performing the assay in situ, for example, or in other suitable conditions, a single pair of label extender probes may be designed to contain the entire complement to the target sequence (half of which would be encoded in the L-1 sequence of a first label extender probe, and the other half of which would be encoded in the second L-1 sequence of the second label extender probe).

Appropriate combinations of the amount of complementarity between the label extenders and the component of the label probe system, number of label extenders binding to the component, and stringency of hybridization can, for example, be determined experimentally by one of skill in the art. For example, a particular number of label extenders and a particular set of hybridization conditions can be selected, while the number of nucleotides of complementarity between the label extenders and the component is varied until hybridization of the label extenders to a nucleic acid captures the component to the nucleic acid while hybridization of a single label extender does not efficiently capture the component. Stringency can be controlled, for example, by controlling the formamide concentration, chaotropic salt concentration, salt concentration, pH, organic solvent content, and/or hybridization temperature.

As noted, the Tₘ of any nucleic acid duplex can be directly measured, using techniques well known in the art. For example, a thermal denaturation curve can be obtained for the duplex, the midpoint of which corresponds to the Tₘ. It will be evident that such denaturation curves can be obtained under conditions having essentially any relevant pH, salt concentration, solvent content, and/or the like.

The Tₘ for a particular duplex (e.g., an approximate Tₘ) can also be calculated. For example, the Tₘ for an oligonucleotide-target duplex can be estimated using the following algorithm, which incorporates nearest neighbor thermodynamic parameters: Tm (Kelvin)=ΔH°/(ΔS°+R lnCₜ), where the changes in standard enthalpy (ΔH°) and entropy (ΔS°) are calculated from nearest neighbor thermodynamic parameters (see, e.g., SantaLucia (1998) “A unified view of polymer, dumbbell, and oligonucleotide DNA nearest-neighbor thermodynamics” Proc. Natl. Acad. Sci. USA 95:1460-1465, Sugimoto et al. (1996) “Improved thermodynamic parameters and helix initiation factor to predict stability of DNA duplexes” Nucleic Acids Research 24: 4501-4505, Sugimoto et al. (1995) “Thermodynamic parameters to predict stability of RNA/DNA hybrid duplexes”

Biochemistry 34:11211-11216, and et al. (1998) “Thermodynamic parameters for an expanded nearest-neighbor model for formation of RNA duplexes with Watson-Crick base pairs” Biochemistry 37: 14719-14735), R is the ideal gas constant (1.987 cal·K^{-l}mole⁻¹), and Cₜ is the molar concentration of the oligonucleotide. The calculated Tₘ is optionally corrected for salt concentration, e.g., Na⁺ concentration, using the formula

1/Tₘ(Na⁺)=1/Tₘ(1M)+(4.29f(G·C)−3.95)×10⁻⁵ ln [Na⁺]+9.40×10⁻⁶ ln²[Na⁺].

### See, e.g., Owczarzy et al. (2004) “Effects of Sodium Ions on DNA Duplex Oligomers:

Improved Predictions of Melting Temperatures” Biochemistry 43:3537-3554 for further details. A Web calculator for estimating Tₘ using the above algorithms is available on the Internet at scitools.idtdna.com/analyzer/oligocalc.asp. Other algorithms for calculating Tₘ are known in the art and are optionally applied to the present invention.

Typically, the component of the label probe system (e.g., the amplification multimer or preamplifier) is capable of hybridizing simultaneously to two of the m label extenders in a subset, although it optionally hybridizes to three, four, or more of the label extenders. In one class of embodiments, e.g., embodiments in which two (or more) label extenders bind to the component of the label probe system, sequence L-2 is 20 nucleotides or less in length. For example, L-2 can be between 9 and 17 nucleotides in length, e.g., between 12 and 15 nucleotides in length, between 13 and 15 nucleotides in length, or between 13 and 14 nucleotides in length. As noted, m is at least two, and can be at least three, at least five, at least 10, or more. m can be the same or different from subset to subset of label extenders.

The label extenders can be configured in any of a variety ways. For example, the two label extenders that hybridize to the component of the label probe system can assume a cruciform arrangement, with one label extender having L-1 5′ of L-2 and the other label extender having L-1 3′ of L-2. Unexpectedly, however, a configuration in which either the 5′ or the 3′ end of both label extenders hybridizes to the nucleic acid while the other end binds to the component yields stronger binding of the component to the nucleic acid than does a cruciform arrangement of the label extenders. Thus, in one class of embodiments, the at least two label extenders (e.g., the m label extenders in a subset) each have L-1 5′ of L-2 or each have L-1 3′ of L-2. For example, L-1, which hybridizes to the nucleic acid of interest, can be at the 5′ end of each label extender, while L-2, which hybridizes to the component of the label probe system, is at the 3′ end of each label extender (or vice versa). L-1 and L-2 are optionally separated by additional sequence. In one exemplary embodiment, L-1 is located at the 5′ end of the label extender and is about 20-30 nucleotides in length, L-2 is located at the 3′ end of the label extender and is about 13-14 nucleotides in length, and L-1 and L-2 are separated by a spacer (e.g., 5 Ts).

A label extender, preamplifier, amplification multimer, label probe, capture probe and/or capture extender optionally comprises at least one non-natural nucleotide. For example, a label extender and the component of the label probe system (e.g., the amplification multimer or preamplifier) optionally comprise, at complementary positions, at least one pair of non-natural nucleotides that base pair with each other but that do not Watson-Crick base pair with the bases typical to biological DNA or RNA (i.e., A, C, G, T, or U). Examples of normatural nucleotides include, but are not limited to, Locked NucleicAcid™ nucleotides (available from Exiqon A/S, (www.)exiqon.com; see, e.g., SantaLucia Jr. (1998) Proc Natl Acad Sci 95:1460-1465) and isoG, isoC, and other nucleotides used in the AEGIS system (Artificially Expanded Genetic Information System, available from EraGen Biosciences, (www.)eragen.com; see, e.g., U.S. Pat. No. 6,001,983, U.S. Pat. No. 6,037,120, and U.S. Pat. No. 6,140,496). Use of such non-natural base pairs (e.g., isoG-isoC base pairs) in the probes can, for example, reduce background and/or simplify probe design by decreasing cross hybridization, or it can permit use of shorter probes (e.g., shorter sequences L-2 and M-1) when the non-natural base pairs have higher binding affinities than do natural base pairs.

The methods can optionally be used to quantitate the amounts of the nucleic acids of interest present in the sample. For example, in one class of embodiments, an intensity of a signal from the label is measured, e.g., for each subset of particles or selected position on the solid support, and correlated with a quantity of the corresponding nucleic acid of interest present.

As noted, blocking probes are optionally also hybridized to the nucleic acids of interest, which can reduce background in the assay. For a given nucleic acid of interest, the corresponding label extenders, optional capture extenders, and optional blocking probes are preferably complementary to physically distinct, nonoverlapping sequences in the nucleic acid of interest, which are preferably, but not necessarily, contiguous. The Tₘs of the capture extender-nucleic acid, label extender-nucleic acid, and blocking probe-nucleic acid complexes are preferably greater than the temperature at which the capture extenders, label extenders, and/or blocking probes are hybridized to the nucleic acid, e.g., by 5° C. or 10° C. or preferably by 15° C. or more, such that these complexes are stable at that temperature. Potential CE and LE sequences (e.g., potential sequences C-3 and L-1) are optionally examined for possible interactions with non-corresponding nucleic acids of interest, LEs or CEs, the preamplifier, the amplification multimer, the label probe, and/or any relevant genomic sequences, for example; sequences expected to cross-hybridize with undesired nucleic acids are typically not selected for use in the CEs or LEs. See, e.g., Player et al. (2001) “Single-copy gene detection using branched DNA (bDNA) in situ hybridization” J Histochem Cytochem 49:603-611 and U.S. patent application 60/680,976. Examination can be, e.g., visual (e.g., visual examination for complementarity), computational (e.g., computation and comparison of binding free energies), and/or experimental (e.g., cross-hybridization experiments). Capture probe sequences are preferably similarly examined, to ensure that the polynucleotide sequence C-1 complementary to a particular capture probe's sequence C-2 is not expected to cross-hybridize with any of the other capture probes that are to be associated with other subsets of particles or selected positions on the support.

At any of various steps, materials not captured on the solid support are optionally separated from the support. For example, after the capture extenders, nucleic acids, label extenders, blocking probes, and support-bound capture probes are hybridized, the support is optionally washed to remove unbound nucleic acids and probes; after the label extenders and amplification multimer are hybridized, the support is optionally washed to remove unbound amplification multimer; and/or after the label probes are hybridized to the amplification multimer, the support is optionally washed to remove unbound label probe prior to detection of the label.

In embodiments in which different nucleic acids are captured to different subsets of particles, one or more of the subsets of particles is optionally isolated, whereby the associated nucleic acid of interest is isolated. Similarly, nucleic acids can be isolated from selected positions on a spatially addressable solid support. The isolated nucleic acid can optionally be removed from the particles and/or subjected to further manipulation, if desired (e.g., amplification by PCR or the like).

As another exemplary embodiment, determining which subsets of particles have a nucleic acid of interest captured on the particles may further comprise amplifying any nucleic acid of interest captured on the particles. A wide variety of techniques for amplifying nucleic acids are known in the art, including, but not limited to, PCR (polymerase chain reaction), rolling circle amplification, and transcription mediated amplification. (See, e.g., Hatch et al. (1999) “Rolling circle amplification of DNA immobilized on solid surfaces and its application to multiplex mutation detection” Genet Anal. 15:35-40; Baner et al. (1998) “Signal amplification of padlock probes by rolling circle replication,” Nucleic Acids Res., 26:5073-8; and Nallur et al. (2001) “Signal amplification by rolling circle amplification on DNA microarrays,” Nucleic Acids Res., 29:E118.) A labeled primer and/or labeled nucleotides are optionally incorporated during amplification. In other embodiments, the nucleic acids of interest captured on the particles are detected and/or amplified without identifying the subsets of particles and/or the nucleic acids (e.g., in embodiments in which the subsets of particles are not distinguishable).

The methods can be used to detect the presence of the nucleic acids of interest in essentially any type of sample. For example, the sample can be derived from an animal, a human, a plant, a cultured cell, a virus, a bacterium, a pathogen, and/or a microorganism. The sample optionally includes a cell lysate, an intercellular fluid, a bodily fluid (including, but not limited to, blood, serum, saliva, urine, sputum, or spinal fluid), and/or a conditioned culture medium, and is optionally derived from a tissue (e.g., a tissue homogenate), a biopsy, and/or a tumor. Similarly, the nucleic acids can be essentially any desired nucleic acids (e.g., DNA, RNA, mRNA, rRNA, miRNA, etc.). As just a few examples, the nucleic acids of interest can be derived from one or more of an animal, a human, a plant, a cultured cell, a microorganism, a virus, a bacterium, or a pathogen.

Due to cooperative hybridization of multiple target capture probes to a nucleic acid of interest, for example, even nucleic acids present at low concentration can be captured. Thus, in one class of embodiments, at least one of the nucleic acids of interest is present in the sample in a non-zero amount of 200 attomole (amol) or less, 150 amol or less, 100 amol or less, 50 amol or less, 10 amol or less, 1 amol or less, or even 0.1 amol or less, 0.01 amol or less, 0.001 amol or less, or 0.0001 amol or less. Similarly, two nucleic acids of interest can be captured simultaneously, even when they differ in concentration by 1000-fold or more in the sample. The methods are thus extremely versatile.

Capture of a particular nucleic acid is optionally quantitative. Thus, in one exemplary class of embodiments, the sample includes a first nucleic acid of interest, and at least 30%, at least 50%, at least 80%, at least 90%, at least 95%, or even at least 99% of a total amount of the first nucleic acid present in the sample is captured on a first subset of particles. Second, third, etc. nucleic acids can similarly be quantitatively captured. Such quantitative capture can occur without capture of a significant amount of undesired nucleic acids, even those of very similar sequence to the nucleic acid of interest.

As noted, the methods can be used for gene expression analysis. Accordingly, in one class of embodiments, the two or more nucleic acids of interest comprise two or more mRNAs. The methods can also be used for clinical diagnosis and/or detection of microorganisms, e.g., pathogens. Thus, in certain embodiments, the nucleic acids include bacterial and/or viral genomic RNA and/or DNA (double-stranded or single-stranded), plasmid or other extra-genomic DNA, or other nucleic acids derived from microorganisms (pathogenic or otherwise). It will be evident that double-stranded nucleic acids of interest will typically be denatured before hybridization with capture extenders, label extenders, and the like.

The methods may similarly be applied towards detection and identification of single nucleotide polymorphisms (SNPs) residing in a genomic sample. The methods are very flexible and can be applied equally as well to SNP detection across the entire genome, if desired. Various methods of SNP detection may be employed, as explained in further detail below.

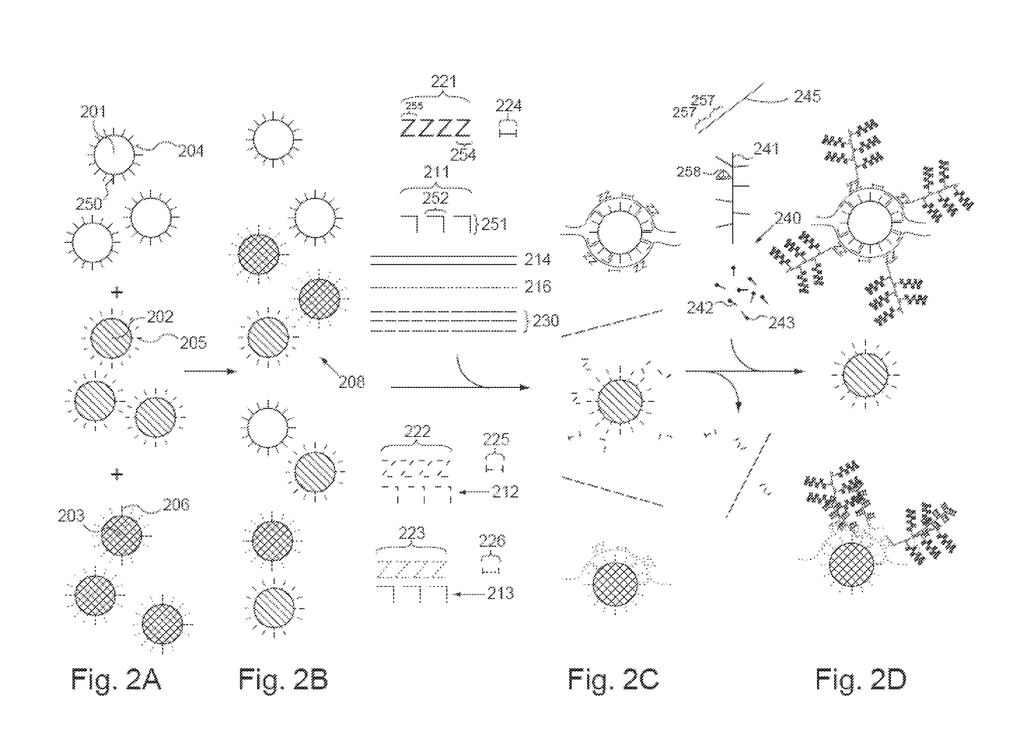

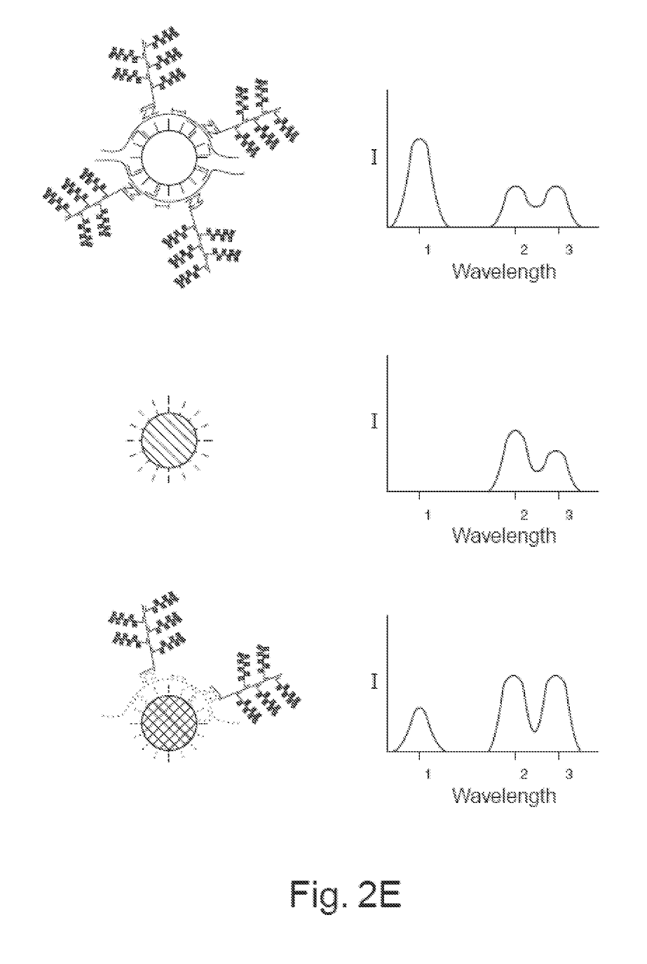

An exemplary embodiment is schematically illustrated in FIG. 2. Panel A illustrates three distinguishable subsets of microspheres 201, 202, and 203, which have associated therewith capture probes 204, 205, and 206, respectively. Each capture probe includes a sequence C-2 (250), which is different from subset to subset of microspheres. The three subsets of microspheres are combined to form pooled population 208 (Panel B). A subset of capture extenders is provided for each nucleic acid of interest; subset 211 for nucleic acid 214, subset 212 for nucleic acid 215 which is not present, and subset 213 for nucleic acid 216. Each capture extender includes sequences C-1 (251, complementary to the respective capture probe's sequence C-2) and C-3 (252, complementary to a sequence in the corresponding nucleic acid of interest). Three subsets of label extenders (221, 222, and 223 for nucleic acids 214, 215, and 216, respectively) and three subsets of blocking probes (224, 225, and 226 for nucleic acids 214, 215, and 216, respectively) are also provided. Each label extender includes sequences L-1 (254, complementary to a sequence in the corresponding nucleic acid of interest) and L-2 (255, complementary to M-1). Non-target nucleic acids 230 are also present in the sample of nucleic acids.

Subsets of label extenders 221 and 223 are hybridized to nucleic acids 214 and 216, respectively. In addition, nucleic acids 214 and 216 are hybridized to their corresponding subset of capture extenders (211 and 213, respectively), and the capture extenders are hybridized to the corresponding capture probes (204 and 206, respectively), capturing nucleic acids 214 and 216 on microspheres 201 and 203, respectively (Panel C). Materials not bound to the microspheres (e.g., capture extenders 212, nucleic acids 230, etc.) are separated from the microspheres by washing. Label probe system 240 including preamplifier 245 (which includes two sequences M-1 257), amplification multimer 241 (which includes sequences M-2 258), and label probe 242 (which contains label 243) is provided. Each preamplifier 245 is hybridized to two label extenders, amplification multimers 241 are hybridized to the preamplifier, and label probes 242 are hybridized to the amplification multimers (Panel D). Materials not captured on the microspheres are optionally removed by washing the microspheres. Microspheres from each subset are identified, e.g., by their fluorescent emission spectrum (λ₂ and λ₃, Panel E), and the presence or absence of the label on each subset of microspheres is detected (λ₁, Panel E). Since each nucleic acid of interest is associated with a distinct subset of microspheres, the presence of the label on a given subset of microspheres correlates with the presence of the corresponding nucleic acid in the original sample.

As depicted in FIG. 2, all of the label extenders in all of the subsets typically include an identical sequence L-2. Optionally, however, different label extenders (e.g., label extenders in different subsets) can include different sequences L-2. Also as depicted in FIG. 2, each capture probe typically includes a single sequence C-2 and thus hybridizes to a single capture extender. Optionally, however, a capture probe can include two or more sequences C-2 and hybridize to two or more capture extenders. Similarly, as depicted, each of the capture extenders in a particular subset typically includes an identical sequence C-1, and thus only a single capture probe is needed for each subset of particles; however, different capture extenders within a subset optionally include different sequences C-1 (and thus hybridize to different sequences C-2, within a single capture probe or different capture probes on the surface of the corresponding subset of particles).

In the embodiment depicted in FIG. 2, the label probe system includes the preamplifier, amplification multimer, and label probe. It will be evident that similar considerations apply to embodiments in which the label probe system includes only an amplification multimer and label probe or only a label probe.

The various hybridization and capture steps can be performed simultaneously or sequentially, in any convenient order. For example, in embodiments in which capture extenders are employed, each nucleic acid of interest can be hybridized simultaneously with its corresponding subset of m label extenders and its corresponding subset of n capture extenders, and then the capture extenders can be hybridized with capture probes associated with the solid support. Materials not captured on the support are preferably removed, e.g., by washing the support, and then the label probe system is hybridized to the label extenders.

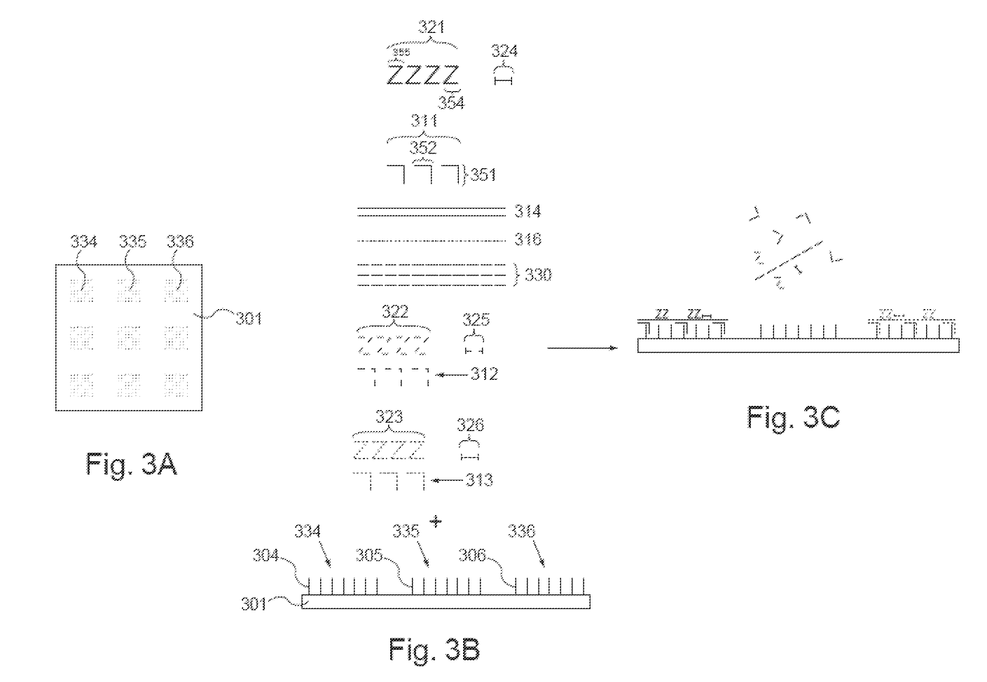

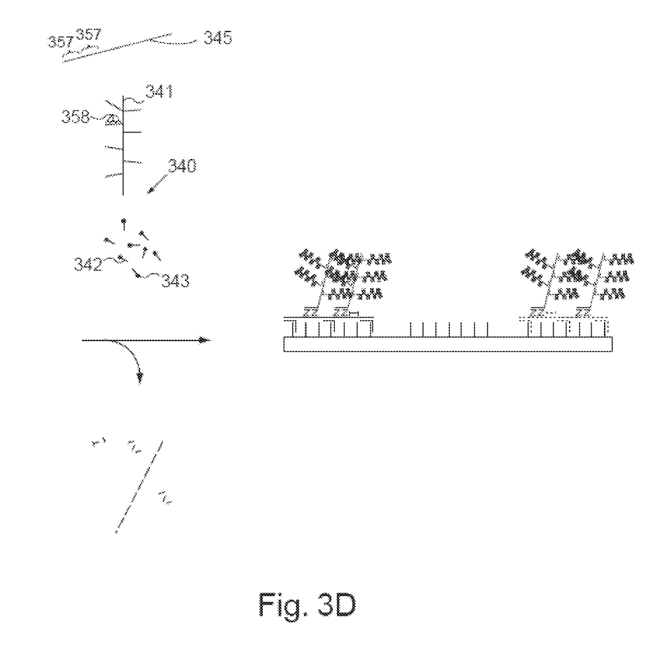

Another exemplary embodiment is schematically illustrated in FIG. 3. Panel A depicts solid support 301 having nine capture probes provided on it at nine selected positions (e.g., 334-336). Panel B depicts a cross section of solid support 301, with distinct capture probes 304, 305, and 306 at different selected positions on the support (334, 335, and 336, respectively). A subset of capture extenders is provided for each nucleic acid of interest. Only three subsets are depicted; subset 311 for nucleic acid 314, subset 312 for nucleic acid 315 which is not present, and subset 313 for nucleic acid 316. Each capture extender includes sequences C-1 (351, complementary to the respective capture probe's sequence C-2) and C-3 (352, complementary to a sequence in the corresponding nucleic acid of interest). Three subsets of label extenders (321, 322, and 323 for nucleic acids 314, 315, and 316, respectively) and three subsets of blocking probes (324, 325, and 326 for nucleic acids 314, 315, and 316, respectively) are also depicted (although nine would be provided, one for each nucleic acid of interest). Each label extender includes sequences L-1 (354, complementary to a sequence in the corresponding nucleic acid of interest) and L-2 (355, complementary to M-1). Non-target nucleic acids 330 are also present in the sample of nucleic acids.

Subsets of label extenders 321 and 323 are hybridized to nucleic acids 314 and 316, respectively. Nucleic acids 314 and 316 are hybridized to their corresponding subset of capture extenders (311 and 313, respectively), and the capture extenders are hybridized to the corresponding capture probes (304 and 306, respectively), capturing nucleic acids 314 and 316 at selected positions 334 and 336, respectively (Panel C). Materials not bound to the solid support (e.g., capture extenders 312, nucleic acids 330, etc.) are separated from the support by washing. Label probe system 340 including preamplifier 345 (which includes two sequences M-1 357), amplification multimer 341 (which includes sequences M-2 358) and label probe 342 (which contains label 343) is provided. Each preamplifier 345 is hybridized to two label extenders, amplification multimers 341 are hybridized to the preamplifier, and label probes 342 are hybridized to the amplification multimers (Panel D). Materials not captured on the solid support are optionally removed by washing the support, and the presence or absence of the label at each position on the solid support is detected. Since each nucleic acid of interest is associated with a distinct position on the support, the presence of the label at a given position on the support correlates with the presence of the corresponding nucleic acid in the original sample.

Another general class of embodiments provides methods of detecting one or more nucleic acids, using the novel label extender configuration described above. In the methods, a sample comprising or suspected of comprising the nucleic acids of interest, one or more subsets of m label extenders, wherein m is at least two, and a label probe system are provided. Each subset of m label extenders is capable of hybridizing to one of the nucleic acids of interest. The label probe system comprises a label, and a component of the label probe system (e.g., a preamplifier or an amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2.

Those nucleic acids of interest present in the sample are captured on a solid support. Each nucleic acid of interest captured on the solid support is hybridized to its corresponding subset of m label extenders, and the label probe system (or the component thereof) is hybridized to the m label extenders at a hybridization temperature. The hybridization temperature is greater than a melting temperature Tₘ of a complex between each individual label extender and the component of the label probe system. The presence or absence of the label on the solid support is then detected. Since the label is associated with the nucleic acid(s) of interest via hybridization of the label extenders and label probe system, the presence or absence of the label on the solid support is correlated with the presence or absence of the nucleic acid(s) of interest on the solid support and thus in the original sample.

Typically, the one or more nucleic acids of interest comprise two or more nucleic acids of interest, and the one or more subsets of m label extenders comprise two or more subsets of m label extenders.

In one class of embodiments in which the one or more nucleic acids of interest comprise two or more nucleic acids of interest and the one or more subsets of m label extenders comprise two or more subsets of m label extenders, a pooled population of particles which constitute the solid support is provided. The population comprises two or more subsets of particles, and a plurality of the particles in each subset is distinguishable from a plurality of the particles in every other subset. (Typically, substantially all of the particles in each subset are distinguishable from substantially all of the particles in every other subset.) The particles in each subset have associated therewith a different capture probe.

Two or more subsets of n capture extenders, wherein n is at least two, are also provided. Each subset of n capture extenders is capable of hybridizing to one of the nucleic acids of interest, and the capture extenders in each subset are capable of hybridizing to one of the capture probes, thereby associating each subset of n capture extenders with a selected subset of the particles. Each of the nucleic acids of interest present in the sample is hybridized to its corresponding subset of n capture extenders and the subset of n capture extenders is hybridized to its corresponding capture probe, thereby capturing the nucleic acid on the subset of particles with which the capture extenders are associated.

Typically, in this class of embodiments, at least a portion of the particles from each subset are identified and the presence or absence of the label on those particles is detected. Since a correlation exists between a particular subset of particles and a particular nucleic acid of interest, which subsets of particles have the label present indicates which of the nucleic acids of interest were present in the sample.

In other embodiments in which the one or more nucleic acids of interest comprise two or more nucleic acids of interest and the one or more subsets of m label extenders comprise two or more subsets of m label extenders, the nucleic acids are captured at different positions on a non-particulate, spatially addressable solid support. Thus, in one class of embodiments, the solid support comprises two or more capture probes, wherein each capture probe is provided at a selected position on the solid support. Two or more subsets of n capture extenders, wherein n is at least two, are provided. Each subset of n capture extenders is capable of hybridizing to one of the nucleic acids of interest, and the capture extenders in each subset are capable of hybridizing to one of the capture probes, thereby associating each subset of n capture extenders with a selected position on the solid support. Each of the nucleic acids of interest present in the sample is hybridized to its corresponding subset of n capture extenders and the subset of n capture extenders is hybridized to its corresponding capture probe, thereby capturing the nucleic acid on the solid support at the selected position with which the capture extenders are associated.

Typically, in this class of embodiments, the presence or absence of the label at the selected positions on the solid support is detected. Since a correlation exists between a particular position on the support and a particular nucleic acid of interest, which positions have a label present indicates which of the nucleic acids of interest were present in the sample.

Essentially all of the features noted for the methods above apply to these embodiments as well, as relevant; for example, with respect to composition of the label probe system; type of label; type of solid support; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

In one aspect, the invention provides methods for capturing a labeled probe to a target nucleic acid, through hybridization of the labeled probe directly to label extenders hybridized to the nucleic acid or through hybridization of the labeled probe to one or more nucleic acids that are in turn hybridized to the label extenders.

Accordingly, one general class of embodiments provides methods of capturing a label to a first nucleic acid of interest in a multiplex assay in which two or more nucleic acids of interest are to be detected. In the methods, a sample comprising the first nucleic acid of interest and also comprising or suspected of comprising one or more other nucleic acids of interest is provided. A first subset of m label extenders, wherein m is at least two, and a label probe system comprising the label are also provided. The first subset of m label extenders is capable of hybridizing to the first nucleic acid of interest, and a component of the label probe system is capable of hybridizing simultaneously to at least two of the m label extenders in the first subset. The first nucleic acid of interest is hybridized to the first subset of m label extenders, and the label probe system is hybridized to the m label extenders, thereby capturing the label to the first nucleic acid of interest.

Essentially all of the features noted for the embodiments above apply to these methods as well, as relevant; for example, with respect to configuration of the label extenders, number of label extenders per subset, composition of the label probe system, type of label, number of nucleic acids of interest, source of the sample and/or nucleic acids, and/or the like. For example, in one class of embodiments, the label probe system comprises a label probe, which label probe comprises the label, and which label probe is capable of hybridizing simultaneously to at least two of the m label extenders. In other embodiments, the label probe system includes the label probe and an amplification multimer that is capable of hybridizing simultaneously to at least two of the m label extenders. Similarly, in yet other embodiments, the label probe system includes the label probe, an amplification multimer, and a preamplifier that is capable of hybridizing simultaneously to at least two of the m label extenders.

Another general class of embodiments provides methods of capturing a label to a nucleic acid of interest. In the methods, m label extenders, wherein m is at least two, are provided. The m label extenders are capable of hybridizing to the nucleic acid of interest. A label probe system comprising the label is also provided. A component of the label probe system is capable of hybridizing simultaneously to at least two of the m label extenders. Each label extender comprises a polynucleotide sequence L-1 that is complementary to a polynucleotide sequence in the nucleic acid of interest and a polynucleotide sequence L-2 that is complementary to a polynucleotide sequence in the component of the label probe system, and the m label extenders each have L-1 5′ of L-2 or wherein the m label extenders each have L-1 3′ of L-2. The nucleic acid of interest is hybridized to the m label extenders, and the label probe system is hybridized to the m label extenders at a hybridization temperature, thereby capturing the label to the nucleic acid of interest. Preferably, the hybridization temperature is greater than a melting temperature Tₘ of a complex between each individual label extender and the component of the label probe system.

Essentially all of the features noted for the embodiments above apply to these methods as well, as relevant; for example, with respect to configuration of the label extenders, number of label extenders per subset, composition of the label probe system, type of label, and/or the like. For example, in one class of embodiments, the label probe system comprises a label probe, which label probe comprises the label, and which label probe is capable of hybridizing simultaneously to at least two of the m label extenders. In other embodiments, the label probe system includes the label probe and an amplification multimer that is capable of hybridizing simultaneously to at least two of the m label extenders. Similarly, in yet other embodiments, the label probe system includes the label probe, an amplification multimer, and a preamplifier that is capable of hybridizing simultaneously to at least two of the m label extenders.

### Compositions

Compositions related to the methods are another feature of the invention. Thus, one general class of embodiments provides a composition for detecting two or more nucleic acids of interest. In one aspect, the composition includes a pooled population of particles. The population comprises two or more subsets of particles, with a plurality of the particles in each subset being distinguishable from a plurality of the particles in every other subset. The particles in each subset have associated therewith a different capture probe. In another aspect, the composition includes a solid support comprising two or more capture probes, wherein each capture probe is provided at a selected position on the solid support.

The composition also optionally may include two or more subsets of n capture extenders, wherein n is at least two, two or more subsets of m label extenders, wherein m is at least two, and a label probe system comprising a label, wherein a component of the label probe system is capable of hybridizing simultaneously to at least two of the m label extenders in a subset. Each subset of n capture extenders is capable of hybridizing to one of the nucleic acids of interest, and the capture extenders in each subset are capable of hybridizing to one of the capture probes and thereby associating each subset of n capture extenders with a selected subset of the particles or with a selected position on the solid support. Similarly, each subset of m label extenders is capable of hybridizing to one of the nucleic acids of interest.

The composition optionally includes a sample comprising or suspected of comprising at least one of the nucleic acids of interest, e.g., two or more, three or more, etc. nucleic acids. Optionally, the composition comprises one or more of the nucleic acids of interest or target nucleic acids. In one class of embodiments, each nucleic acid of interest present in the composition is hybridized to its corresponding subset of n capture extenders, and the corresponding subset of n capture extenders is hybridized to its corresponding capture probe. Each nucleic acid of interest is thus associated with an identifiable subset of the particles. In this class of embodiments, each nucleic acid of interest present in the composition is also hybridized to its corresponding subset of m label extenders. The component of the label probe system (e.g., the amplification multimer or preamplifier) is hybridized to the m label extenders. The composition is maintained at a hybridization temperature that is greater than a melting temperature Tₘ of a complex between each individual label extender and the component of the label probe system (e.g., the amplification multimer or preamplifier). The hybridization temperature is typically about 5° C. or more greater than the Tₘ, e.g., about 7° C. or more, about 10° C. or more, about 12° C. or more, about 15° C. or more, about 17° C. or more, or even about 20° C. or more greater than the T. Where in situ applications are called for, the capture probe, capture extenders and particles are not included in the compositions.

Essentially all of the features noted for the methods above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

Another general class of embodiments provides a composition for detecting one or more nucleic acids of interest. The composition includes a solid support comprising one or more capture probes, one or more subsets of n capture extenders, wherein n is at least two, one or more subsets of m label extenders, wherein m is at least two, and a label probe system comprising a label. Each subset of n capture extenders is capable of hybridizing to one of the nucleic acids of interest, and the capture extenders in each subset are capable of hybridizing to one of the capture probes and thereby associating each subset of n capture extenders with the solid support. Each subset of m label extenders is capable of hybridizing to one of the nucleic acids of interest. A component of the label probe system (e.g., a preamplifier or amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2.

In one class of embodiments, the one or more nucleic acids of interest comprise two or more nucleic acids of interest, the one or more subsets of n capture extenders comprise two or more subsets of n capture extenders, the one or more subsets of m label extenders comprise two or more subsets of m label extenders, and the solid support comprises a pooled population of particles. The population comprises two or more subsets of particles. A plurality of the particles in each subset are distinguishable from a plurality of the particles in every other subset, and the particles in each subset have associated therewith a different capture probe. The capture extenders in each subset are capable of hybridizing to one of the capture probes and thereby associating each subset of n capture extenders with a selected subset of the particles.

In another class of embodiments, the one or more nucleic acids of interest comprise two or more nucleic acids of interest, or target nucleic acids, the one or more subsets of n capture extenders comprise two or more subsets of n capture extenders, the one or more subsets of m label extenders comprise two or more subsets of m label extenders, and the solid support comprises two or more capture probes, wherein each capture probe is provided at a selected position on the solid support. The capture extenders in each subset are capable of hybridizing to one of the capture probes and thereby associating each subset of n capture extenders with a selected position on the solid support.

Essentially all of the features noted for the embodiments above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

For example, the label probe system can include an amplification multimer or preamplifier, which amplification multimer or preamplifier is capable of hybridizing to the at least two label extenders. The composition optionally includes one or more of the nucleic acids of interest, wherein each nucleic acid of interest is hybridized to its corresponding subset of m label extenders and to its corresponding subset of n capture extenders, which in turn is hybridized to its corresponding capture probe. The amplification multimer or preamplifier is hybridized to the m label extenders. The composition is maintained at a hybridization temperature that is greater than a melting temperature Tₘ of a complex between each individual label extender and the amplification multimer or preamplifier (e.g., about 5° C. or more, about 7° C. or more, about 10° C. or more, about 12° C. or more, about 15° C. or more, about 17° C. or more, or about 20° C. or more greater than the Tₘ).

Compositions are also understood to comprise label extenders and capture extenders having one or more nucleic acid analogs. That is, the sequences of L-1 and C-3, may contain anywhere from 1% to 100% nucleic acid analogs, such as, for instance, cEt, LNA, PNA and the like, and mixtures thereof. With regard to cEt, it is understood that other nucleic acid analogs of similar structure and having the same or similar properties, i.e. the ability to increase the melting temperature of a hybridization event between the capture extender and/or label extender sequence and the target sequence. Thus, minor alterations to the structure of the cEt, including, but not limited to, addition of other alkyl groups, alkylene groups, thiols, amines, carboxyls, etc. which have similar chemical properties suitable to the assays and methods provided above, are also included in these compositions. Compositions are further intended to include those compositions designed specifically for detection of target nucleic acids in situ, which would not require the use of, and therefore not include in the composition, capture probes, capture extenders and/or particles.

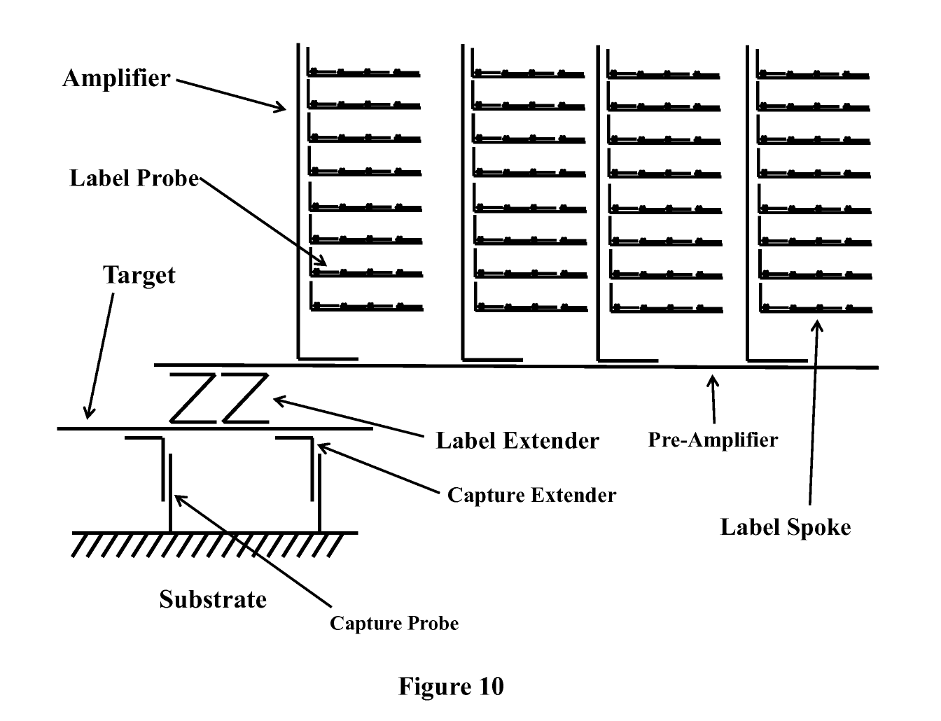

Compositions will also in some embodiments comprise all of the components of the label probe system as depicted in FIG. 10, including different sets of distinguishable label probes, each addressable to different sets of label spoke probes, amplifier probes and pre-amplifier probes. In various embodiments, the compositions will comprise as many different sets of label probe systems as desired for assaying targets at a desired plex level.

### Kits

Yet another general class of embodiments provides a kit for detecting two or more nucleic acids of interest or two or proteins of interest, or both. In one aspect, the kit optionally includes a pooled population of particles. The population comprises two or more subsets of particles, with a plurality of the particles in each subset being distinguishable from a plurality of the particles in every other subset. The particles in each subset have associated therewith a different capture probe, or are otherwise capable of having immobilized thereon one or more samples containing target proteins or target nucleic acids. In another aspect, the kit includes a solid support comprising two or more capture probes, wherein each capture probe is provided at a selected position on the solid support.

The kit may also includes two or more subsets of n capture extenders, wherein n is at least two, two or more subsets of m label extenders, wherein m is at least two, and a label probe system comprising a label, wherein a component of the label probe system is capable of hybridizing simultaneously to at least two of the m label extenders in a subset. Each subset of n capture extenders is capable of hybridizing to one of the nucleic acids of interest, and the capture extenders in each subset are capable of hybridizing to one of the capture probes and thereby associating each subset of n capture extenders with a selected subset of the particles or with a selected position on the solid support. Similarly, each subset of m label extenders is capable of hybridizing to one of the nucleic acids of interest. The components of the kit are packaged in one or more containers. The kit optionally also includes instructions for using the kit to capture and detect the nucleic acids of interest, one or more buffered solutions (e.g., lysis buffer, diluent, hybridization buffer, and/or wash buffer), standards comprising one or more nucleic acids at known concentration, and/or the like.

Essentially all of the features noted for the embodiments above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

Another general class of embodiments provides a kit for detecting one or more nucleic acids of interest or one or more protein of interest. The kit may optionally include a solid support comprising one or more capture probes, one or more subsets of n capture extenders, wherein n is at least two, one or more subsets of m label extenders, wherein m is at least two, and a label probe system comprising a label. Each subset of n capture extenders is capable of hybridizing to one of the nucleic acids of interest, and the capture extenders in each subset are capable of hybridizing to one of the capture probes and thereby associating each subset of n capture extenders with the solid support. Each subset of m label extenders is capable of hybridizing to one of the nucleic acids of interest. A component of the label probe system (e.g., a preamplifier or amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2. The components of the kit are packaged in one or more containers. The kit optionally also includes instructions for using the kit to capture and detect the nucleic acids of interest, one or more buffered solutions (e.g., lysis buffer, diluent, hybridization buffer, and/or wash buffer), standards comprising one or more nucleic acids at known concentration, and/or the like.

Essentially all of the features noted for the embodiments above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

For example, in one class of embodiments, the one or more nucleic acids of interest comprise two or more nucleic acids of interest, the one or more subsets of n capture extenders comprise two or more subsets of n capture extenders, the one or more subsets of m label extenders comprise two or more subsets of m label extenders, and the solid support comprises a pooled population of particles. The population comprises two or more subsets of particles. A plurality of the particles in each subset are distinguishable from a plurality of the particles in every other subset, and the particles in each subset have associated therewith a different capture probe. The capture extenders in each subset are capable of hybridizing to one of the capture probes and thereby associating each subset of n capture extenders with a selected subset of the particles.

In another class of embodiments, the one or more nucleic acids of interest comprise two or more nucleic acids of interest, the one or more subsets of n capture extenders comprise two or more subsets of n capture extenders, the one or more subsets of m label extenders comprise two or more subsets of m label extenders, and the solid support comprises two or more capture probes, wherein each capture probe is provided at a selected position on the solid support. The capture extenders in each subset are capable of hybridizing to one of the capture probes and thereby associating each subset of n capture extenders with a selected position on the solid support.

Kits are also understood to comprise label extenders and capture extenders having one or more nucleic acid analogs. That is, the sequences of L-1 and C-3, may contain anywhere from 1% to 100% nucleic acid analogs, such as, for instance, cEt, LNA, PNA and the like, and mixtures thereof. With regard to cEt, it is understood that other nucleic acid analogs of similar structure and having the same or similar properties, i.e. the ability to increase the melting temperature of a hybridization event between the capture extender and/or label extender sequence and the target sequence. Thus, minor alterations to the structure of the cEt, including, but not limited to, addition of other alkyl groups, alkylene groups, thiols, amines, carboxyls, etc. which have similar chemical properties suitable to the assays and methods provided above, are also included in these kits. Kits are further intended to include those compositions designed specifically for detection of target nucleic acids in situ, which would not require the use of, and therefore not include in the kit, capture probes, capture extenders and/or particles.

Kits will also in some embodiments comprise all of the components of the label probe system as depicted in FIG. 10, including different sets of distinguishable label probes, each addressable to different sets of label spoke probes, amplifier probes and pre-amplifier probes. In various embodiments, the kits will comprise as many different sets of label probe systems as desired for assaying targets at a desired plex level.

### Systems

In one aspect, the invention includes systems, e.g., systems used to practice the methods herein and/or comprising the compositions described herein. The system can include, e.g., a fluid and/or microsphere handling element, a fluid and/or microsphere containing element, a laser for exciting a fluorescent label and/or fluorescent microspheres, a detector for detecting light emissions from a chemiluminescent reaction or fluorescent emissions from a fluorescent label and/or fluorescent microspheres, and/or a robotic element that moves other components of the system from place to place as needed (e.g., a multiwell plate handling element). For example, in one class of embodiments, a composition of the invention is contained in a flow cytometer, a Luminex 100™ or HTS™ instrument, a microplate reader, a microarray reader, a luminometer, a colorimeter, fluorescence micropscope, substrates (such as slides, well plates, etc.) on which samples may be prepared for assay, or like instrument.

The system can optionally include a computer. The computer can include appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for controlling the operation of components of the system (e.g., for controlling a fluid handling element, robotic element and/or laser). The computer can also receive data from other components of the system, e.g., from a detector, and can interpret the data, provide it to a user in a human readable format, or use that data to initiate further operations, in accordance with any programming by the user.

### Labels

A wide variety of labels are well known in the art and can be adapted to the practice of the present invention. For example, luminescent labels and light-scattering labels (e.g., colloidal gold particles) have been described. (See, e.g., Csaki et al. (2002) “Gold nanoparticles as novel label for DNA diagnostics,” Expert Rev. Mol. Diagn., 2:187-93).

As another example, a number of fluorescent labels are well known in the art, including but not limited to, hydrophobic fluorophores (e.g., phycoerythrin, rhodamine, Alexa Fluor 488 and fluorescein), green fluorescent protein (GFP) and variants thereof (e.g., cyan fluorescent protein and yellow fluorescent protein), and quantum dots. (See, e.g., The Handbook: A Guide to Fluorescent Probes and Labeling Technologies, Tenth Edition or Web Edition (2006) from Invitrogen (available on the internet at probes.invitrogen.com/handbook), for descriptions of fluorophores emitting at various different wavelengths (including tandem conjugates of fluorophores that can facilitate simultaneous excitation and detection of multiple labeled species). For use of quantum dots as labels for biomolecules, see e.g., Dubertret et al. (2002) Science, 298:1759; Nature Biotechnology (2003) 21:41-46; and Nature Biotechnology (2003) 21:47-51. Other various labels are known in the art, such as Alexa Fluor Dyes (Life Technologies, Inc., California, USA, available in a wide variety of wavelengths, see for instance, Panchuk, et al., J. Hist. Cyto., 47:1179-1188, 1999), biotin-based dyes, digoxigenin, AttoPhos (JBL Scientific, Inc., California, USA, available in a variety of wavelengths, see for instance, Cano et al., Biotechniques, 12(2):264-269, 1992), etc.

Labels can be introduced to molecules, e.g. polynucleotides, during synthesis or by postsynthetic reactions by techniques established in the art; for example, kits for fluorescently labeling polynucleotides with various fluorophores are available from Molecular Probes, Inc. ((www.)molecularprobes.com), and fluorophore-containing phosphoramidites for use in nucleic acid synthesis are commercially available. Similarly, signals from the labels (e.g., absorption by and/or fluorescent emission from a fluorescent label) can be detected by essentially any method known in the art. For example, multicolor detection, detection of FRET, fluorescence polarization, and the like, are well known in the art.

### Microspheres

Microspheres are preferred particles in certain embodiments described herein since they are generally stable, are widely available in a range of materials, surface chemistries and uniform sizes, and can be fluorescently dyed. Microspheres can be distinguished from each other by identifying characteristics such as their size (diameter) and/or their fluorescent emission spectra, for example. Furthermore, as explained in better detail above, the particles may be microspheres which may also be microparticles having a code therein.

Luminex Corporation ((www.)luminexcorp.com), for example, offers 100 sets of uniform diameter polystyrene microspheres. The microspheres of each set are internally labeled with a distinct ratio of two fluorophores. A flow cytometer or other suitable instrument can thus be used to classify each individual microsphere according to its predefined fluorescent emission ratio. Fluorescently-coded microsphere sets are also available from a number of other suppliers, including Radix Biosolutions ((www.)radixbiosolutions.com) and Upstate Biotechnology ((www.)upstatebiotech.com). Alternatively, BD Biosciences ((www.)bd.com) and Bangs Laboratories, Inc. ((www.) bangslabs.com) offer microsphere sets distinguishable by a combination of fluorescence and size. As another example, microspheres can be distinguished on the basis of size alone, but fewer sets of such microspheres can be multiplexed in an assay because aggregates of smaller microspheres can be difficult to distinguish from larger microspheres.

Microspheres with a variety of surface chemistries are commercially available, from the above suppliers and others (e.g., see additional suppliers listed in Kellar and Iannone (2002) “Multiplexed microsphere-based flow cytometric assays” Experimental Hematology 30:1227-1237 and Fitzgerald (2001) “Assays by the score” The Scientist 15[11]:25). For example, microspheres with carboxyl, hydrazide or maleimide groups are available and permit covalent coupling of molecules (e.g., polynucleotide capture probes with free amine, carboxyl, aldehyde, sulfhydryl or other reactive groups) to the microspheres. As another example, microspheres with surface avidin or streptavidin are available and can bind biotinylated capture probes; similarly, microspheres coated with biotin are available for binding capture probes conjugated to avidin or streptavidin. In addition, services that couple a capture reagent of the customer's choice to microspheres are commercially available, e.g., from Radix Biosolutions ((www.)radixbiosolutions.com).

Protocols for using such commercially available microspheres (e.g., methods of covalently coupling polynucleotides to carboxylated microspheres for use as capture probes, methods of blocking reactive sites on the microsphere surface that are not occupied by the polynucleotides, methods of binding biotinylated polynucleotides to avidin-functionalized microspheres, and the like) are typically supplied with the microspheres and are readily utilized and/or adapted by one of skill. In addition, coupling of reagents to microspheres is well described in the literature. For example, see Yang et al. (2001) “BADGE, Beads Array for the Detection of Gene Expression, a high-throughput diagnostic bioassay” Genome Res. 11:1888-98; Fulton et al. (1997) “Advanced multiplexed analysis with the FlowMetrix™ system” Clinical Chemistry 43:1749-1756; Jones et al. (2002) “Multiplex assay for detection of strain-specific antibodies against the two variable regions of the G protein of respiratory syncytial virus” 9:633-638; Camilla et al. (2001) “Flow cytometric microsphere-based immunoassay: Analysis of secreted cytokines in whole-blood samples from asthmatics” Clinical and Diagnostic Laboratory Immunology 8:776-784; Martins (2002) “Development of internal controls for the Luminex instrument as part of a multiplexed seven-analyte viral respiratory antibody profile” Clinical and Diagnostic Laboratory Immunology 9:41-45; Kellar and Iannone (2002) “Multiplexed microsphere-based flow cytometric assays” Experimental Hematology 30:1227-1237; Oliver et al. (1998) “Multiplexed analysis of human cytokines by use of the FlowMetrix system” Clinical Chemistry 44:2057-2060; Gordon and McDade (1997) “Multiplexed quantification of human IgG, IgA, and IgM with the FlowMetrix™ system” Clinical Chemistry 43:1799-1801; U.S. Pat. No. 5,981,180 entitled “Multiplexed analysis of clinical specimens apparatus and methods” to Chandler et al. (Nov. 9, 1999); U.S. Pat. No. 6,449,562 entitled “Multiplexed analysis of clinical specimens apparatus and methods” to Chandler et al. (Sep. 10, 2002); and references therein.

Methods of analyzing microsphere populations (e.g. methods of identifying microsphere subsets by their size and/or fluorescence characteristics, methods of using size to distinguish microsphere aggregates from single uniformly sized microspheres and eliminate aggregates from the analysis, methods of detecting the presence or absence of a fluorescent label on the microsphere subset, and the like) are also well described in the literature. See, e.g., the above references.

Suitable instruments, software, and the like for analyzing microsphere populations to distinguish subsets of microspheres and to detect the presence or absence of a label (e.g., a fluorescently labeled label probe) on each subset are commercially available. For example, flow cytometers are widely available, e.g., from Becton-Dickinson ((www.) bd.com) and Beckman Coulter ((www.)beckman.com). Luminex 100™ and Luminex HTS™ systems (which use microfluidics to align the microspheres and two lasers to excite the microspheres and the label) are available from Luminex Corporation ((www.) luminexcorp.com); the similar Bio-Plex™ Protein Array System is available from Bio-Rad Laboratories, Inc. ((www.)bio-rad.com). A confocal microplate reader suitable for microsphere analysis, the FMAT™ System 8100, is available from Applied Biosystems ((www.)appliedbiosystems.com).

As another example of particles that can be adapted for use in the present invention, sets of microbeads that include optical barcodes are available from CyVera Corporation ((www.)cyvera.com). The optical barcodes are holographically inscribed digital codes that diffract a laser beam incident on the particles, producing an optical signature unique for each set of microbeads.

### Polynucleotide Synthesis

Methods of making nucleic acids (e.g., by in vitro amplification, purification from cells, or chemical synthesis), methods for manipulating nucleic acids (e.g., by restriction enzyme digestion, ligation, etc.) and various vectors, cell lines and the like useful in manipulating and making nucleic acids are described in the above references. In addition, methods of making branched polynucleotides (e.g., amplification multimers) are described in U.S. Pat. No. 5,635,352, U.S. Pat. No. 5,124,246, U.S. Pat. No. 5,710,264, and U.S. Pat. No. 5,849,481, as well as in other references mentioned above.

In addition, essentially any polynucleotide (including, e.g., labeled or biotinylated polynucleotides) can be custom or standard ordered from any of a variety of commercial sources, such as The Midland Certified Reagent Company ((www.)mcrc.com), The Great American Gene Company ((www.)genco.com), ExpressGen Inc. ((www.) expressgen.com), Qiagen (oligos.qiagen.com) and many others.

A label, biotin, or other moiety can optionally be introduced to a polynucleotide, either during or after synthesis. For example, a biotin phosphoramidite can be incorporated during chemical synthesis of a polynucleotide. Alternatively, any nucleic acid can be biotinylated using techniques known in the art; suitable reagents are commercially available, e.g., from Pierce Biotechnology ((www.)piercenet.com). Similarly, any nucleic acid can be fluorescently labeled, for example, by using commercially available kits such as those from Molecular Probes, Inc. ((www.) molecularprobes.com) or Pierce Biotechnology ((www.)piercenet.com) or by incorporating a fluorescently labeled phosphoramidite during chemical synthesis of a polynucleotide.

### Signal Amplification

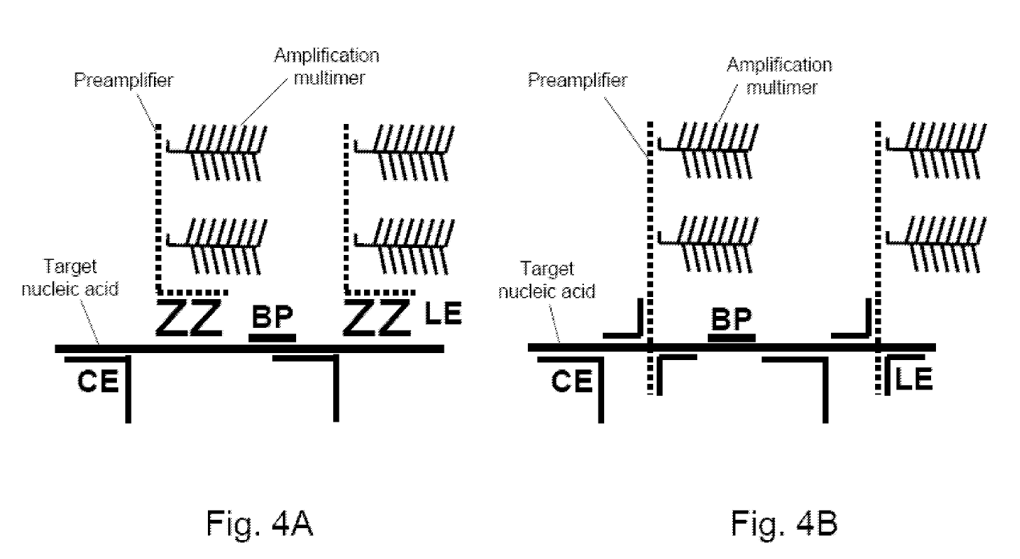

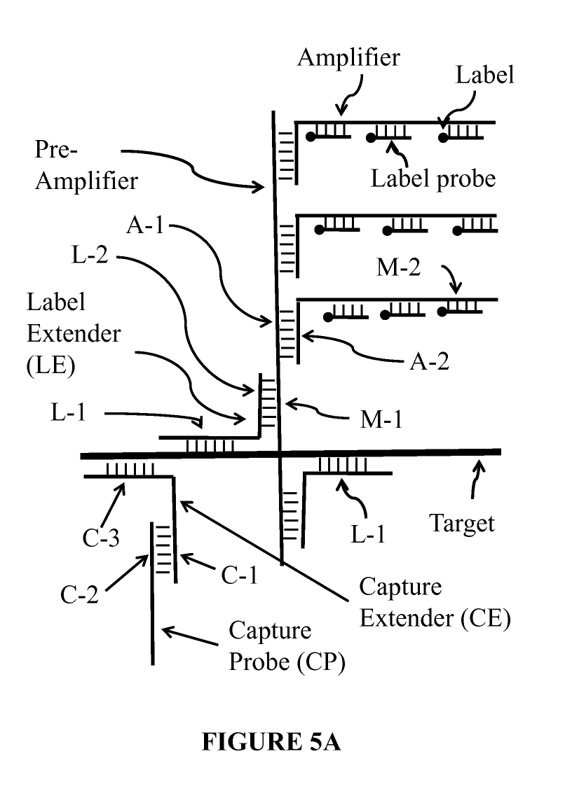

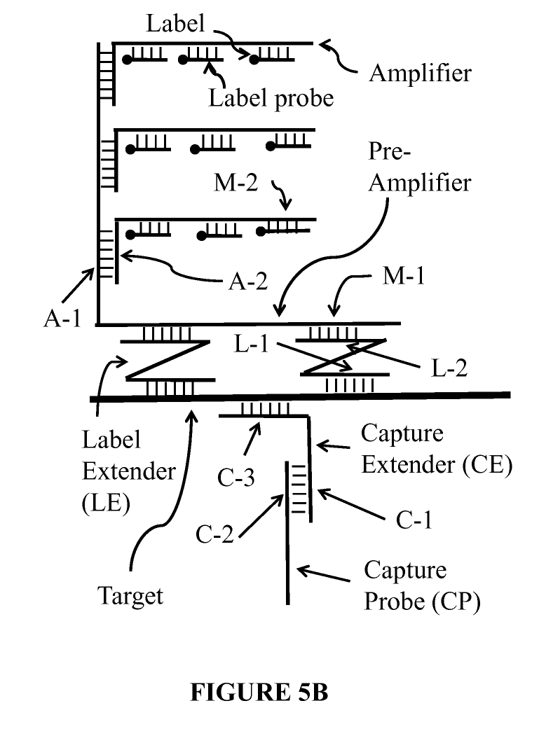

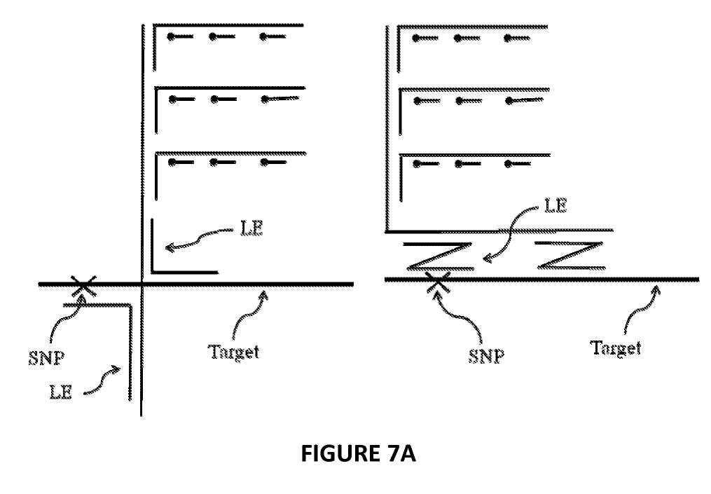

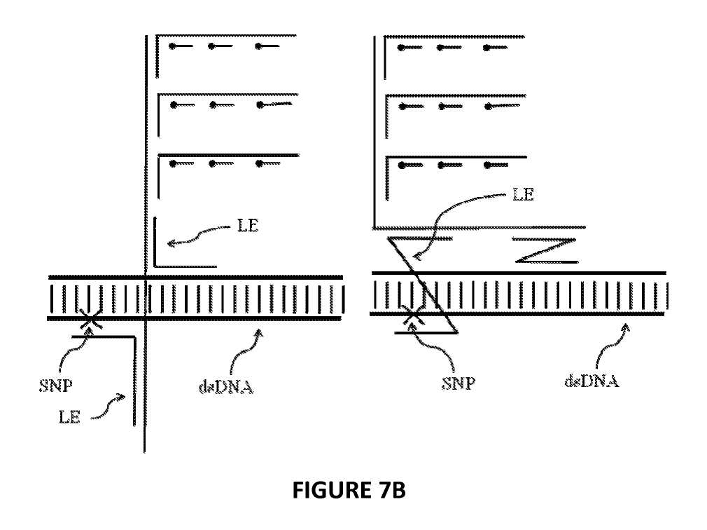

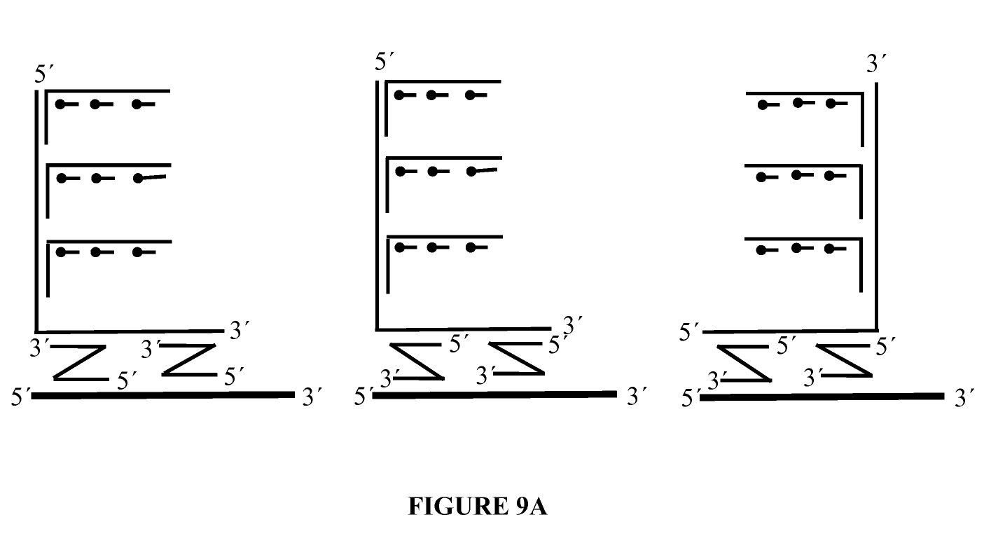

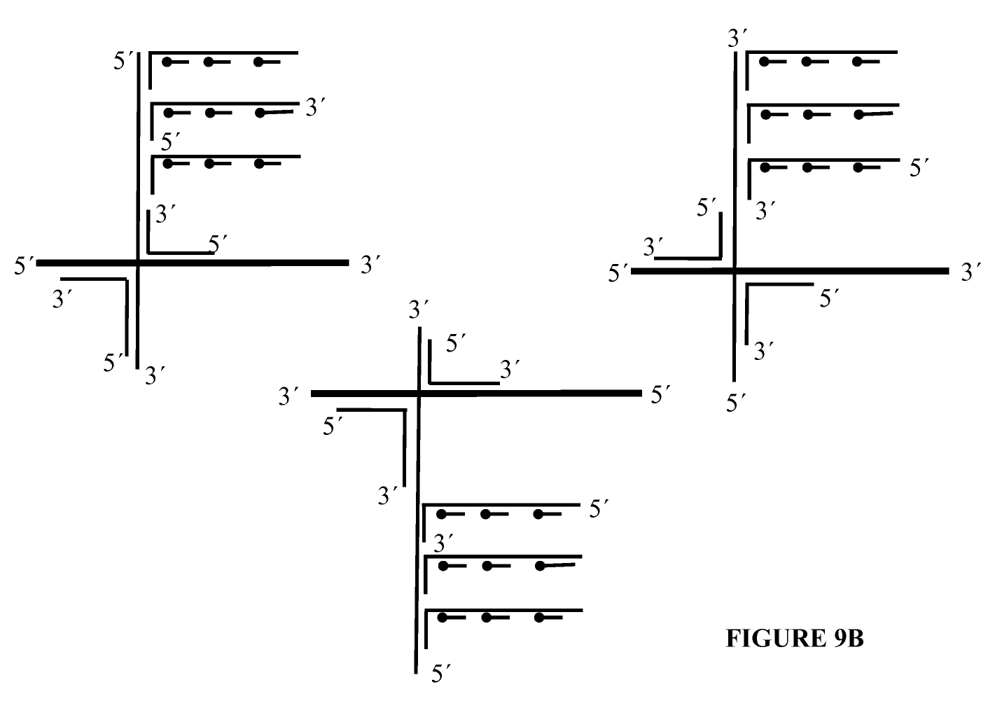

Amplification of signal is achieved by hybridization of nucleic acid probe structures in a scaffolding manner such that the number of label probes, and hence the number of labels, that can bind is increased. For instance, referring to FIG. 10, the pre-amplifier and amplifier structures depicted in FIGS. 4, 5, 7 and 9 can be increased even further by designing additional probes on which additional label probes may hybridize.

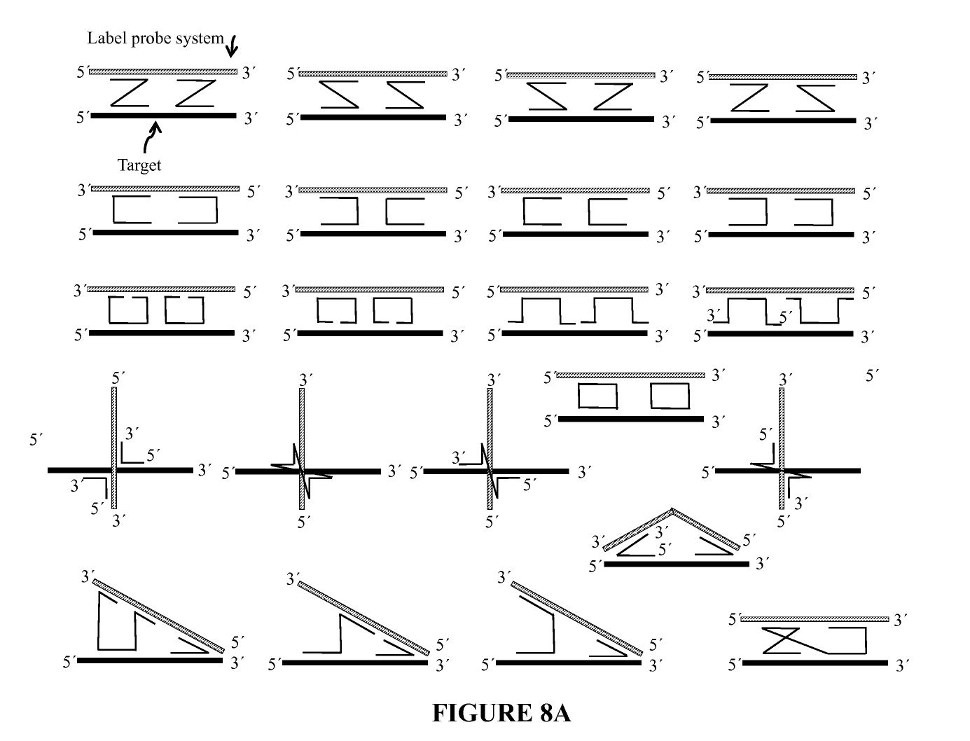

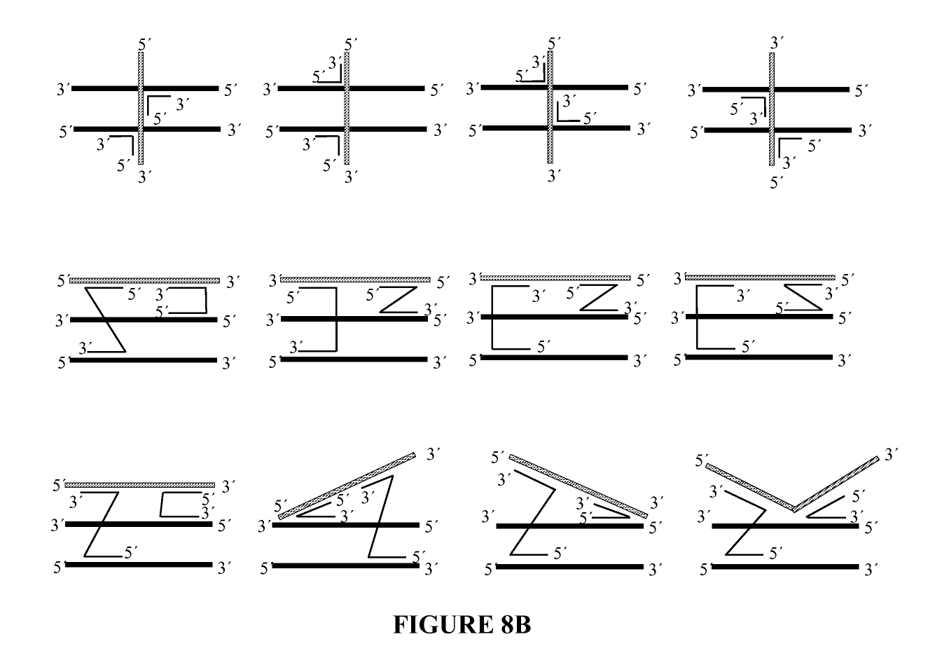

FIG. 10 provides an illustration of the type of signal amplification achievable by the present methods. Though FIG. 10 depicts the target nucleic acid being captured to a substrate using the cooperative hybridization strategy, this is an optional feature. The label probe system depicted in FIG. 10 may be used on free targets as well as targets conjugated or otherwise associated to a substrate, as illustrated. FIG. 10 provides labels for the usual components of capture probes, capture extenders and label extenders. It is noted that generally any suitable label extender probe geometry may be utilized in this context, such as, but not limited to, those LE geometries depicted in FIGS. 8A and 8B. Likewise, target nucleic acids may be single stranded or double stranded.

The pre-amplifier depicted in FIG. 10 may be any desired length, to accommodate as many amplifiers as desired. Likewise, the length of amplifier may be shorter or longer depending on the number of label spokes desired to hybridize to each amplifier. Finally, each label spoke length may be varied depending on the number label probe hybridization sequences incorporated into the label spoke probe.

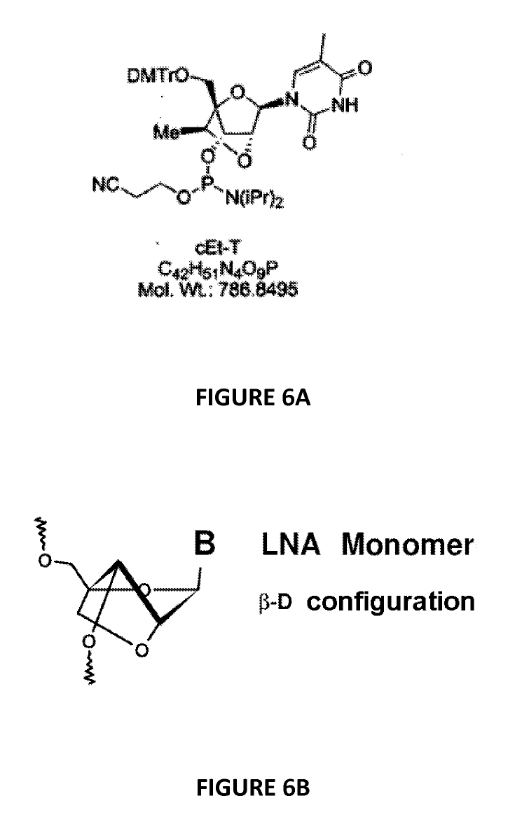

Any of the hybridization points on the scaffold depicted in FIG. 10, such as the L-1 sequences, A-1 sequences, C-1 sequences, and the like, may be substituted with nucleic acid analogs. For instance, nucleic acid analogs such as constrained-ethyl (cEt) analogs may be used, as depicted in FIG. 6A. (See, for additional variations of this analog which may also be suitable in the present embodiments, Seth et al., “Short Antisense Oligonucleotides with Novel 2′-4′ Conformationaly Restricted Nucleoside Analogues Show Improved Potency Without Increased Cytotoxicity in Animals,” J. Med. Chem., 52(1):10-13, 2009, incorporated herein by reference in its entirety for all purposes). The capture extender probe may be entirely comprised of such cEt analogs, or may be only partially comprised of cEt analogs. Specifically, the capture extender probe may only have cEt analogs at sequence L-1. The capture extender may have cEt analogs at the C-2 sequence as well as the L-1 sequence and/or cEt content beyond those sequences up to and including the entire capture extender probe. Use of the cEt analogs in the capture portion of the assay is especially beneficial because it is known that cEt analogs, when present in probes, act to increase the melting temperature of the resulting hybridized probe:target pair, which provides increased stability of the hybridized pair and therefore increased stability of the captured target nucleic acid bound to the encoded microparticle.

Any of the probes depicted in FIG. 10, such as, but not limited to, the label extender probes. For example, the length of label extender probes may vary in length anywhere from 10 to 60 nucleic acids or more, i.e. 11, 13, 15, 17, 19, 21, 25, 30, 35, 40, 45 or 50 nucleic acids in length. The sequence L-1 will also vary depending on the identity of the target and the number of potentially cross-reacting probes within the hybridization mixture. For instance, L-1 may be anywhere from 7 to 50 nucleic acids in length, or 10 to 40, or 12 to 30 or 15 to 20 nucleotides in length. The sequence L-1 may be entirely comprised of nucleic acid analogs or only partly comprised of nucleic acid analogs. For instance, it may be that every other nucleic acid is an analog in L-1, providing a 50% substitution of analog for native or wild type base. Alternatively, the L-1 sequence may be 100% comprised of nucleic acid analog. Further the L-1 sequence may be 10%, 20%, 30%, 40%, 50%, 60%, 70%, 80% or 90% comprised of nucleic acid analog. The underlying principle to the use of nucleotide analogs, such as cEt, is to increase the melting temperature or temperature at which the L-1 sequence remains hybridized to the target sequence. Typically, the LE and CE may be designed such that the target melting temperature for the assay is in the range of 50° C. to 56° C., or 49° C. to 57° C., or 48° C. to 48° C., etc. However, this may vary depending on buffer conditions and assay. For instance, when performing an in situ assay, it may be useful to add a neutralizing or denaturing agent such as formamide, and thereafter adjust the target melting temperature downwards to a range of 40° C. to 50° C. or lower. Thus the amount of melting temperature-increasing nucleotide analog present in L-1 can be doped up or down to the desired and empirically-determined most suitable amount to achieve the desired melting temperature, which will in turn provide the best performance with respect to affinity and specificity. Further, the desired melting temperature may also be target-dependant. That is, if a specific target nucleic acid is rich, or has a high content of, G and C bases, then perhaps less melting temperature-increasing nucleic acid analogs, like cEt, will be necessary to achieve the desired melting temperature, as compared to a target region which is rich in A and T bases. In summary, design of the L-1 sequence, as in any probe sequence binding to the target, and determination of the amount of nucleotide analog to use in a specific embodiment of the presently disclosed assays, will depend on many factors including target sequence, buffer conditions and melting temperature needed to achieve the desired specificity and affinity in the assay.

Such signal amplification strategies may be universally applied to any number of assays, including, but not limited to, assays involving identification and/or detection of target proteins. For instance, an antibody may be conjugated to a pre-amplifier probe. The antibody could then be used to bind to antigen which is immobilized onto a substrate. Measurement of a signal using the label probe system depicted in FIG. 10 would then be indicative of the presence and quantity of target protein in the sample.

Target nucleic acids may be, but are not limited to, ssDNA, dsDNA, RNA, mRNA, siRNA, miRNA (premature and mature), rRNA, tRNA and the like. Assays may be conducted in vitro (as depicted in FIG. 10), in cellulo, or in situ, using various known techniques and available products. The labels associated with the label probe system may be any of a variety of useful and detectable labels known in the art, as explained in further detail above.

### Amplifier Flexing

In addition to amplifying signal, the present embodiments lend themselves to increasing the number of targets that may be simultaneously assayed, i.e. multiplexing. That is, it is possible to design label probe systems that bind a single type of label, as explained in detail above, or alternatively a label probe system such as that depicted in FIG. 10, or other such similar systems, may be designed to bind to multiple different distinguishable labels. For instance, in some cases it is desirable to increase the number of targets to as many as 100 such targets per single cell or tissue sample.

Disclosed are two embodiments which achieve this goal. One embodiment is generally directed to varying the size, shape and color of various microspheres, beads or microparticles used to capture targets. The second embodiment is generally directed to varying the design of the label probe system such that different combinations of labels may be bound to the label spokes or amplifiers, and the like.

In the first embodiment, the target nucleic acids are bound to a substrate. The substrate may be, for instance, a bead, microparticle, or microsphere. The size and shape of the substrate may be varied any number of ways. For instance, current methodologies employ differently colored beads onto which are bound target nucleic acids by use of a capture probe and capture extender. However, this limited set of beads may be increased by simply varying the size of the bead and/or the shape of the bead. Beads may be oblong, oval, square, triangular, rhomboid, octagonal, etc.

In the second embodiment, a different sequence is used to hybridized to each different label probe by each amplifier. Thus, each label probe system has a unique set of amplifiers, each comprising a nucleic acid sequence that specifically hybridizes to a unique set of label probes. This system can be further manipulated such that each label probe system can hybridize as many as two, three, or four or even more different label probes so that each label probe system can comprise a combination of fluorophores, for instance, such that each label probe system associated with each different target will emit a uniquely identifiable signal.

For instance, as depicted in FIG. 11, various labels may be associated with various targets. In the top left illustration, all the label probes are of one type (white circle). Moving to the next illustration to the right of the first illustration, there is shown another embodiment where the label probe system may be a mixture of two different labels (white circles and grey circles). This mixture, through FRET or other interactions, may provide a signal which is distinguishable from the signal associated with the white signal depiction and distinguishable from the second signal associated with the grey circle depiction, thus creating a third unique signal when the two are combined into a single label probe system. Further illustrated in FIG. 11 are black circles, which are meant to represent a third type of label that, when combined with others, produces additional unique and distinguishable signals. The illustration on the top right of FIG. 11 shows a label probe system mixing all three labels which could produce yet a different signal.

In one non-limiting embodiment, these different signals may be different wavelengths of absorption as measured by fluorescence microscopy. Various fluorescent dyes are available which, when mixed together in close proximity on a molecular scale such as depicted in FIG. 11, produce a veritable rainbow of colors from all points of the fluorescent spectrum. The number of unique combinations of signals is only limited by the ability to detect each separate signal. Thus, given the appropriate number of filters applied to fluorescent microscope instrumentation, a wide variety of different signals may be detected, each indicative of a different target.

It should further be evident from FIG. 11 that although the depiction shows the cruciform geometry for the label extender probes, any other suitable label probe geometry may be utilized. Furthermore, though only four amplifiers are shown per each label probe system, any number of amplifiers may be utilized to provide greater signal amplification. Label probe systems such as that depicted in FIG. 10 may be used which provides many more options for attachment of different families or types of label probes.

Label probes may additionally be mixed and matched as to type of label. For instance fluorescent dyes may be used for one label probe system, while radiolabeled label probes may be used for a second label probe system. And yet still a third label probe system may be comprised of quantum dot type dyes. Further, these amplifier plexing embodiments may be performed in any type of assay, such as an in vitro, in cellulo or in situ assay where the target may be optionally immobilized onto a substrate by means known in the art. Further, the target may be nucleic acids, proteins or even mixtures of both. In embodiments where proteins are the target, the pre-amplifier will simply be conjugated to the antibody specific for the target protein. The label probe system may then be hybridized to the antibody. Optional washes will remove those antibodies that did not bind to target.

In another embodiment of this type, sequential hybridizations and washes may be conducted to achieve a multiplex effect for the label probe systems. For instance, if only two different labels are available, various different label extenders may be synthesized which comprise sequences complimentary to a first set of two different target nucleic acids. The two different target nucleic acids may then be detected by binding of the two different label extenders and then binding thereto two different pre-amplifier and label probe systems, each having a unique and distinguishable label. Once these two targets are detected, the sample, such as, but not limited to, a well plate comprising tissue culture cells, or an FFPE fixed tissue sample, etc., may be washed to remove the hybridized label probe system. After complete washing, a second set of two different label extenders, comprising complementary sequences of a second set of two different target nucleic acids, may then be hybridized to the sample as above, and the labeling procedure repeated. This may be repeated any number of times, in any number of multiples of label extender probes, to achieve the desired multiplex effect.

In another embodiment, the differently colored or shaped substrates described above may be combined with the different label probe systems to achieve additional levels of multiplexing. Instrumentation exists which is able to distinguish differently colored substrate beads. Instrumentation also exists which is able to detect different labels. Thus one single bead color could have associated with it any number of different labels. A matrix could be set forth which matches bead color and label color to yield the identity of the target to be detected. For example, with ten differently colored beads and ten different labels, it is possible to achieve a multiplex of 100.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of amplifying a nucleic acid detection signal, which comprises:
hybridizing one or more label extender probes to a target nucleic acid;
hybridizing a pre-amplifier to the one or more label extender probes;
hybridizing one or more amplifiers to the pre-amplifier;
hybridizing one or more label spoke probes to the one or more amplifiers; and
hybridizing one or more label probes to the one or more label spoke probes.

2. The method according to claim 1, wherein the one or more label probes comprise one or more different labels.

3. The method according to claim 1, wherein the one or more label extender probes comprise one or more nucleic acid analogs.

4. The method according to claim 3, wherein the one or more nucleic acid analogs is cEt.

5. The method according to claim 1, which further comprises:
hybridizing one or more capture extender probes with the target nucleic acid; and
hybridizing the one or more capture extenders to one or more capture probes, wherein the capture probes are covalently attached to a substrate.

6. The method according to claim 5, wherein the substrate comprises one or more substrates of varying size, shape and color.

7. The method according to claim 6, wherein each one of the one or more substrates of varying size, shape and color is each associated with a different target nucleic acid.

8. The method according to claim 1, wherein the method is conducted in situ or in vitro.

9. The method according to claim 1, which further comprises:
providing a target protein;
hybridizing to the target protein a molecule possessing affinity for the target protein, wherein the molecule comprises covalently conjugated thereto a pre-amplifier probe;
hybridizing one or more amplifiers to the pre-amplifier;
hybridizing one or more label spoke probes to the one or more amplifiers; and
hybridizing one or more label probes to the one or more label spoke probes.

10. A method of amplifying a protein detection signal, which comprises:
hybridizing a molecule possessing affinity for the target protein, wherein the molecule comprises covalently conjugated thereto a pre-amplifier probe;
hybridizing one or more amplifiers to the pre-amplifier;
hybridizing one or more label spoke probes to the one or more amplifiers; and
hybridizing one or more label probes to the one or more label spoke probes.

11. The method according to claim 10, wherein the one or more label probes comprise one or more different labels.

12. The method according to claim 10, wherein the target protein is immobilized on a substrate.

13. The method according to claim 12, wherein the substrate comprises one or more substrates of varying size, shape and color.

14. The method according to claim 13, wherein each one of the one or more substrates of varying size, shape and color is each associated with a different target nucleic acid.

15. The method according to claim 10, wherein the method is conducted in situ or in vitro.
